(19) United States
(12) Patent Application Publication
(10) Pub. No.: US 2024/0366471 A1
(43) Pub. Date: Nov. 7, 2024

(54) MULTIDOSE ANTIBODY DRUG PRODUCTS USING PHENOL OR BENZYL ALCOHOL

(71) Applicant: Regeneron Pharmaceuticals, Inc., Tarrytown, NY (US)

(72) Inventors: Xiaolin Tang, Old Tappan, NJ (US); Leonid Breydo, Bronx, NY (US); Julie Porter, Mohegan Lake, NY (US)

(21) Appl. No.: 18/651,719

(22) Filed: May 1, 2024

Related U.S. Application Data
(60) Provisional application No. 63/463,179, filed on May 1, 2023.

(51) Int. Cl. A61J 1/05; A61K 39/00; A61K 47/10

(52) U.S. Cl. CPC A61J 1/05; A61K 47/10; A61K 2039/505

(57) ABSTRACT

Methods for stabilizing an Fc-containing protein preparation in an aqueous solution are provided herein. The disclosed methods can stabilize the Fc-containing protein in a drug product, especially for liquid formulations, by using phenol or benzyl alcohol as preservatives.

### CROSS REFERENCE TO RELATED APPLICATIONS

This application claims the benefit of and priority to U.S. Provisional Application No. 63/463,179 filed on May 1, 2023. The above-referenced application is incorporated herein by reference in its entirety.

### TECHNICAL FIELD OF THE INVENTIONS

The present inventions provide for improved methods of stabilizing an Fc-containing protein preparation in an aqueous solution using preservatives.

### BACKGROUND OF THE INVENTIONS

Multidose antibody drug products are often provided in lyophilized form to extend shelf-life. Some antibody products are provided in solution, which require preservatives. However, preservatives can destabilize antibodies. Furthermore, various antimicrobial preservative agents have been used in marketed biologics, but rarely with monoclonal antibodies (mAb).

In consideration of the impact of typical antimicrobial preservatives on stability of the liquid formulations of several mAbs, it is therefore an object of the invention to provide improved methods for preserving, while stabilizing, an Fc-containing protein preparation in an aqueous solution. It is another object of the invention to provide improved methods for developing formulations containing preservatives with stable Fc-containing proteins in the drug product in multidose Fc-containing protein drug products.

### SUMMARY OF THE INVENTIONS

Methods for stabilizing an Fc-containing protein preparation in an aqueous solution are provided. For example, a multiple-dose container may include a parenteral Fc-containing protein preparation. The preparation may comprise, in an aqueous solution, at least one type of Fc-containing protein. The preparation may also comprise, in the aqueous solution, phenol or benzyl alcohol.

One or more of the following example features may be included. The Fc-containing protein may be at a concentration of about 0.1 mg/ml to about 500 mg/ml. Phenol may be at a concentration of about 1 mg/ml to about 10 mg/ml. Phenol may also be at a concentration of about 2 mg/ml to about 5 mg/ml. Phenol may further be at a concentration of about 3 mg/ml (about 0.3%), for example. Benzyl alcohol may be at a concentration of about 1 mg/ml to about 15 mg/ml. Benzyl alcohol may further be at a concentration of about 3 mg/ml to 12 mg/ml. Benzyl alcohol may further be at a concentration of about 10 mg/ml (about 1%), for example.

The container may be a single patient use container. The container may have a capacity of 1 ml to 100 ml. The container may also have a capacity of 0.5 ml to 100 ml. The container may further have a capacity of 5 ml to 50 ml. The container may even further have a capacity of 20 ml to 40 ml. The container may also have a capacity of 30 ml. The Fc-containing protein may be a monoclonal antibody. The monoclonal antibody may be a bispecific antibody. The container may include one, two, three or more types of Fc-containing proteins. The Fc-containing protein may be a receptor Fc-fusion protein. The Fc-containing protein also may be a trap protein.

In another example implementation, a parenteral Fc-containing protein preparation may include, in an aqueous solution, at least one type of Fc-containing protein. The parenteral Fc-containing protein preparation may also include, in the aqueous solution, phenol or benzyl alcohol.

One or more of the following exemplary features may be included. The Fc-containing protein may be at a concentration of about 0.1 mg/ml to about 500 mg/ml. Phenol may be at a concentration of about 1 mg/ml to about 10 mg/ml. Phenol may also be at a concentration of about 2 mg/ml to about 5 mg/ml. Phenol may further be at a concentration of about 3 mg/ml (about 0.3%), for example. Benzyl alcohol may be at a concentration of about 1 mg/ml to about 15 mg/ml. Benzyl alcohol may further be at a concentration of about 3 mg/ml to about 12 mg/ml. Benzyl alcohol may further be at a concentration of about 10 mg/ml (about 1%), for example. The Fc-containing protein may be a monoclonal antibody. The monoclonal antibody may be a bispecific antibody. The preparation may include one, two, three or more types of Fc-containing proteins. The Fc-containing protein may be a receptor Fc-fusion protein. The Fc-containing protein also may be a trap protein.

Still another example implementation provides a method of stabilizing an Fc-containing protein preparation which may include the step of providing Fc-containing proteins in an aqueous solution containing phenol or benzyl alcohol. The method of stabilizing an Fc-containing protein preparation may also include the step of filling a container with the Fc-containing proteins in an aqueous solution containing phenol or benzyl alcohol.

One or more of the following example features may be included. Phenol or benzyl alcohol may be added to an aqueous solution comprising the Fc-containing proteins. The Fc-containing protein may be at a concentration of about 0.1 mg/ml to about 500 mg/ml. Phenol may be at a concentration of about 1 mg/ml to about 10 mg/ml. Phenol may also be at a concentration of about 2 mg/ml to about 5 mg/ml. Phenol may further be at a concentration of about 3 mg/ml (about 0.3%), for example. Benzyl alcohol may be at a concentration of about 1 mg/ml to about 15 mg/ml. Benzyl alcohol may further be at a concentration of about 3 mg/ml to about 12 mg/ml. Benzyl alcohol may further be at a concentration of about 10 mg/ml (about 1%), for example. The Fc-containing protein may be a monoclonal antibody. The monoclonal antibody may be a bispecific antibody. The preparation may include one, two, three or more types of Fc-containing proteins. The Fc-containing protein may be a receptor Fc-fusion protein. The Fc-containing protein also may be a trap protein.

Further description of the inventions are provided below.

### BRIEF DESCRIPTION OF THE FIGURES

FIGS. 1A-1D depict the effect of preservatives on formulation turbidity under a stress condition. When stored at 45° C. for 3 months, there was relatively more of a turbidity increase observed in formulations containing benzyl alcohol than formulations containing phenol. There was also a different effect observed in different antibody (mAbs) formulations.

FIG. 2A depicts the turbidity (AOD) of antibodies with preservatives at 25° C. No appreciable instability was observed when the drug product was stored at 25° C. for 6 months with tested preservatives. Lines not visible were at zero. FIG. 2B depicts the turbidity (AOD) of antibodies with preservatives at 5° C. No appreciable instability was observed when the drug product was stored at 5° C. for 24 months with tested preservatives. Lines not visible were at zero.

FIGS. 3A-3D depict the effect of preservatives on high molecular weight (HMW) complex formation under stress conditions. When stored at 40° C. for 3 months, there was relatively more of a destabilization effect observed in formulations containing benzyl alcohol than formulations containing phenol. There was also a different effect observed in different antibody (mAbs) formulations.

FIGS. 4A-4D depict the effect on % HMW complex formation from preservatives stored at 25° C. for 6 months. No appreciable destabilization was observed between formulations measuring equivalent amounts of benzyl alcohol versus phenol.

FIG. 5 depicts the effect of antibodies on % HMW complex formation from preservatives stored at 5° C. No appreciable instability was observed when the drug products were stored at 5° C. for 24 months with tested preservatives.

FIGS. 6A-6C depict the effect of preservatives on antibody charge variant formation under stress conditions. No substantial destabilization effect was observed in antibody charge variants with preservatives when the drug product was incubated at 40° C. for 2 months.

FIGS. 7A-7C depict the effect of preservatives on antibody charge variant formation at 25° C. No substantial instability was observed in antibody charge variants with preservatives when the drug product was stored for 6 months at 25° C. with tested preservatives.

FIG. 8A depicts the effect of preservatives on antibody charge variant formation at 5° C. for 12 months. FIG. 8B depicts the effect of preservatives on antibody charge variant formation at 5° C. for 24 months. As seen in FIGS. 8A and 8B, no appreciable instability was observed in antibody charge variants with preservatives when the drug product was stored at 5° C. with tested preservatives.

FIG. 9A depicts the subvisible particle formation for all drug product formulations with or without preservatives when stored at 5° C. for 24 months. Lines not visible were at zero. FIG. 9B depicts the subvisible particle formation for all drug product formulations with or without preservatives when stored at 25° C. for 6 months. Lines not visible were at zero.

FIG. 9C depicts the subvisible particle formation for all drug product formulations with or without preservatives when stored at 40° C. to 3 months. Lines not visible were at zero.

FIG. 10 is a graph concerning the bioburden count (CFU/mL) of Formulation 1. The graph shows the log change in bacteria (E. coli and S. aureus) in the Formulation with 0.3% phenol stored at 20-25° C. over a 28 day period.

FIG. 11 is a graph concerning the bioburden count (CFU/mL) of Formulation 2. The graph shows the log change in bacteria (E. coli and S. aureus) in the formulation with 1% benzyl alcohol stored at 20-25° C. over a 28 day period.

FIG. 12 is a graph concerning the bioburden count (CFU/mL) of Formulation 3. The graph shows the log change in bacteria (E. coli and S. aureus) in the formulation with 0.3% phenol stored at 20-25° C. over a 28 day period.

FIG. 13 is a graph concerning the bioburden count (CFU/mL) of Formulation 4. The graph shows the log change in bacteria (E. coli and S. aureus) in the formulation with 1% benzyl alcohol at 20-25° C. over a 28 day period.

FIG. 14 is a graph concerning the bioburden count (CFU/mL) of Formulation 5. The graph shows the log change in bacteria (E. coli and S. aureus) in the formulation with 0.3% phenol at 20-25° C. over a 28 day period.

FIG. 15 is a graph concerning the bioburden count (CFU/mL) of Formulation 6. The graph shows the log change in bacteria (E. coli and S. aureus) in the formulation with 1% benzyl alcohol at 20-25° C. over a 28 day period.

FIG. 16 is a graph concerning the bioburden count (CFU/mL) of Formulation 7. The graph shows the log change in bacteria (E. coli and S. aureus) in the formulation with 0.3% phenol at 20-25° C. over a 28 day period.

FIG. 17 is a graph concerning the bioburden count (CFU/mL) of Formulation 8. The graph shows the log change in bacteria (E. coli and S. aureus) in the formulation with 1% benzyl alcohol at 20-25° C. over a 28 day period.

### DETAILED DESCRIPTION OF THE INVENTIONS

### I. Definitions

It should be appreciated that this disclosure is not limited to the compositions and methods described herein as well as the experimental conditions described, as such may vary. It is also to be understood that the terminology used herein is for the purpose of describing certain aspects only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

Recitation of ranges of values herein are merely intended to serve as a method of referring individually to each separate value falling within the range, unless otherwise indicated herein, and each separate value is incorporated into the specification as if it were individually recited herein. The preceding ranges are intended to be made clear by context, and no further limitation is implied. All methods described herein can be performed in any suitable order unless otherwise indicated herein or otherwise clearly contradicted by context. The use of any and all examples, or exemplary language (for example, “such as”) provided herein, is intended merely to better illuminate the invention and does not pose a limitation on the scope of the invention unless otherwise claimed. No language in the specification should be construed as indicating any non-claimed element as essential to the practice of the invention

The term “about” in the context of numerical values and ranges refers to values or ranges that approximate or are close to the recited values or ranges such that the invention can perform, such as having a sought rate, amount, density, degree, increase, decrease, percentage, value or presence of a form, variant, temperature or amount of time, as is apparent from the teachings contained herein. Thus, this term encompasses values beyond those simply resulting from systematic error. For example, “about” can signify values either above or below the stated value in a range of approx. +/−10% or more or less depending on the ability to perform.

“Antibody” (mAb) or “antibodies” (mAbs), also referred to as immunoglobulins, are examples of proteins having multiple polypeptide chains and extensive post-translational modifications. Antibodies are often used as therapeutic biomolecules. The canonical immunoglobulin protein (for example, IgG) comprises four polypeptide chains, including two heavy (H) chains and two light (L) chains inter-connected by cysteine disulfide bonds. Each light chain is linked to one heavy chain by one cysteine disulfide bond, and the two heavy chains are bound to each other via two cysteine disulfide bonds. Each heavy chain has a heavy chain variable region (HCVR or VH) and a heavy chain constant region. The heavy chain constant region contains three domains, CH1, CH2 and CH3. Each light chain has a light chain variable region (LCVR or VL) and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3). The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, for example, BIACORE™ or solution-affinity ELISA.

The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. The term antibody also includes bispecific antibody, which includes a heterotetrameric immunoglobulin that can bind to more than one different epitope. Bispecific antibodies are generally described in U.S. Pat. No. 8,586,713, which is incorporated by reference into this application. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014).

“Protein” refers to a molecule comprising two or more amino acid residues joined to each other by a peptide bond. Protein includes polypeptides and peptides and may also include modifications such as glycosylation, lipid attachment, sulfation, gamma-carboxylation of glutamic acid residues, alkylation, hydroxylation and ADP-ribosylation. Proteins can be of scientific or commercial interest, including protein-based drugs, and proteins include, among other things, enzymes, ligands, receptors, antibodies and chimeric or fusion proteins. Proteins are produced by various types of recombinant cells using well-known cell culture methods, and are generally introduced into the cell by genetic engineering techniques (for example, such as a sequence encoding a chimeric protein, or a codon-optimized sequence, an intronless sequence, etc.) where it may reside as an episome or be integrated into the genome of the cell.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

“Fc fusion proteins” comprise part or all of two or more proteins, one of which is an Fc portion of an immunoglobulin molecule, which are not otherwise found together in nature. Preparation of fusion proteins comprising certain heterologous polypeptides fused to various portions of antibody-derived polypeptides (including the Fc domain) has been described, for example, by Ashkenazi et al., Proc. Natl. Acad. ScL USA 88: 10535, 1991; Byrn et al., Nature 344:677, 1990; and Hollenbaugh et al., “Construction of Immunoglobulin Fusion Proteins”, in Current Protocols in Immunology, Suppl. 4, pages 10.19.1-10.19.11, 1992. “Receptor Fc fusion proteins” comprise one or more extracellular domain(s) of a receptor coupled to an Fc moiety, which in some aspects comprises a hinge region followed by a CH2 and CH3 domain of an immunoglobulin. In some aspects, the Fc-fusion protein comprises two or more distinct receptor chains that bind to one or more ligand(s) or other molecules, depending on the type of Fc-fusion protein. For example, an Fc-fusion protein is a Trap, such as for example an IL-1 Trap or VEGF Trap.

A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

The phrase “Fc-containing protein” includes antibodies and Fc-fusion proteins, such as trap proteins, bispecific antibodies, antibody derivatives containing an Fc, antibody fragments containing an Fc, Fc-fusion proteins, immunoadhesins, and other binding proteins that comprise at least a functional portion of an immunoglobulin CH2 and CH3 region. Fc-containing proteins, such as antibodies, can comprise modifications in immunoglobulin domains, including where the modifications affect one or more effector function of the binding protein (for example, modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein (for example, an antibody) and exhibits enhanced serum half-life (as compared with the same Fc-containing protein without the recited modification(s)) and have a modification at position 250 (for example, E or Q); 250 and 428 (for example, L or F); 252 (for example, L/Y/F/W or T), 254 (for example, S or T), and 256 (for example, S/R/Q/E/D or T); or a modification at 428 and/or 433 (for example, L/R/SI/P/Q or K) and/or 434 (for example, H/F or Y); or a modification at 250 and/or 428; or a modification at 307 or 308 (for example, 308F, V308F), and 434. In another example, the modification can comprise a 428L (for example, M428L) and 434S (for example, N434S) modification; a 428L, 2591 (for example, V259I), and a 308F (for example, V308F) modification; a 433K (for example, H433K) and a 434 (for example, 434Y) modification; a 252, 254, and 256 (for example, 252Y, 254T, and 256E) modification; a 250Q and 428L modification (for example, T250Q and M428L); a 307 and/or 308 modification (for example, 308F or 308P).

“Acidic charge variants” are Fc-containing protein (for example, antibody) variants that have a lower pH than the main peak form of the Fc-containing protein. Acidic charge variants tend to have more negative charges.

“Basic charge variants” are Fc-containing protein (for example, antibody) variants that have a higher pH than the main peak form of the Fc-containing protein. Basic charge variants tend to have more positive charges or less negative charges.

“Main peak forms” of Fc-containing proteins (for example, antibodies) are the predominant forms of the Fc-containing protein and have a pH between the acidic charge variants and the basic charge variants.

The phrase “bispecific antibody” includes an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FRl, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild-type organism) appears between two framework regions in a variable region of a light or a heavy chain of an immunoglobulin molecule (for example, an antibody or a T cell receptor). A CDR can be encoded by, for example, a germline sequence or a rearranged or unrearranged sequence, and, for example, by a naive or a mature B cell or a T cell. In some circumstances (for example, for a CDR3), CDRs can be encoded by two or more sequences (for example, germline sequences) that are not contiguous (for example, in a nucleic acid sequence that has not been rearranged) but are contiguous in a B cell nucleic acid sequence, for example, as the result of splicing or connecting the sequences (for example, V-D-J recombination to form a heavy chain CDR3).

“Antibody derivatives and fragments” include, but are not limited to: antibody fragments (for example, ScFv-Fc, dAB-Fc, half antibodies), multispecifics (for example, IgG-ScFv, IgG-dab, ScFV-Fc-ScFV, tri-specific).

“Protein products” refers to the proteins of interest, such as an Fc-containing proteins (for example, antibodies). Protein products can be produced by cells in culture, usually engineered mammalian cells. Typically, the cells in culture, such as in a bioreactor, will produce proteins of interest, and those proteins will become the protein product. The protein product can be subject to later purification, characterization, sterilization, formulation and other finishing steps, such as concentration or lyophilization, and ultimately packaging to form a finished protein product. Proteins products include formulation drug substances (FDS).

The phrase “single-dose drug product” refers to a container designed for use by a single patient as a single injection and/or infusion.

The phrase “single-patient-use container” refers to a parenteral preparation that is intended to be used multiple times for a single patient. For “multiple-dose containers” (as defined below) and single-patient-use containers, the antimicrobial effectiveness testing results will be used to support the labeled beyond-use date (BUD) or discard statements.

The phrase “multiple-dose (multi-dose) drug product” or “multiple-dose (multi-dose) container or vial” refers to a drug-product or container of parenteral preparation that has met antimicrobial effectiveness testing requirements or is excluded from such testing requirements by FDA regulation. A multiple-dose drug product is intended to contain more than one dose of the drug product, for example, a multi-dose vial includes 5-6 doses of the drug product. A multiple-dose drug product for articles or preparations are intended for parenteral administration only and usually contains antimicrobial preservatives. Multiple-dose containers are generally expected to contain 30 mL or less of product. The beyond-use date (BUD) for an opened or entered (for example, needle-punctured) multiple-dose container with antimicrobial preservatives is 28 days, unless otherwise specified by the manufacturer. See, for example, Chapter 51, entitled “Antimicrobial Effectiveness Testing”, of the United States Pharmacopeia (USP).

The phrase “kinetic degradation profiling” refers to the analysis of protein degradation over time (for example, storage stability of the Fc-containing proteins at 5° C. for 24 months, 25° C. for 6 months, 40° C. for 3 months, and 45° C. for 3 months).

The phrase “compounded sterile preparations (CSPs)” or “immediate-use compounded sterile preparations” are sterile drugs that do not contain preservatives and, therefore, are intended for use immediately after opening.

The phrase “cation exchange chromatography (CEX)” is a form of ion exchange chromatography (IEX), which is used to separate molecules based on their net surface charge. Cation exchange chromatography, more specifically, uses a negatively charged ion exchange resin with an affinity for molecules having net positive surface charges.

The phrase “mean or median fluorescence intensity (MFI)” is often used to define and describe the mean intensity and level of antibody expression. The fluorescence intensity indicates how much light (photons) is emitted and it depends on the concentration of an excited fluorophore.

The phrase “Micro-Flow Imaging (MFI)” describes a process used to measure the size distribution, concentration, and morphology of microspheres, protein particulates, silicone droplets and other subvisible/visible particulates using flow microscopy.

The phase “optical density (OD)” refers to turbidity (for example, optical density at 405 nm) of a sample measured using the Molecular Devices SPECTRAmax 190 microplate spectrophotometer.

The phrase “relative humidity (RH)” expressed as a percentage, indicates a present state of absolute humidity relative to a maximum humidity given the same temperature.

The phrase “compendial assays” or “compendial tests” are tests performed to determine if a material's specifications are met and/or to address anticipated regulatory concerns. These test series can also be applied to determine general drug compatibility or for routine quality control.

The phrase “size exclusion-ultra-performance liquid chromatography (SE-UPLC)” or “size exclusion-high-performance liquid chromatography (SE-HPLC)” refers to a high-throughput analytical method, through isocratic condition, to determine and quantify the level of aggregates and fragments of purified antibodies. In other terms, the method separates molecules based on their size and high molecular weight (HMW) by filtration through a gel containing pores of a specific size distribution. Separation occurs when molecules of different sizes are included or excluded from the pores within the matrix of the gel.

The phrase “reversed-phase high-performance liquid chromatography (RP-HPLC)” involves the separation of molecules on the basis of hydrophobicity.

All numerical limits and ranges set forth herein include all numbers or values thereabout or there between of the numbers of the range or limit. The ranges and limits described herein expressly denominate and set forth all integers, decimals and fractional values defined and encompassed by the range or limit.

### II. Methods for Stabilizing Fc-containing Proteins

Multidose antibody drug products are often provided in lyophilized form to extend shelf-life. Some antibody drug products are provided in a solution, which require preservatives to prevent or inhibit microbial growth. However, preservatives may destabilize antibodies in drug products. Therefore, methods for stabilizing antibody drug products requiring preservatives are provided.

The present inventions provide approaches for stabilizing Fc-containing proteins prepared in an aqueous solution and developing formulations containing preservatives to stabilize the Fc-containing protein in the drug product in multidose antibody drug products. Storage stability of the Fc-containing proteins were assessed at various temperatures, as will be described in further detail, including at 5° C., 25° C., 40° C., and 45° C., to enable kinetic degradation profiling, if applicable. One aspect provides a multiple-dose container including at least one type of Fc-containing protein, and a phenol or benzyl alcohol.

Once opened, a container will become contaminated with bacteria and other microorganisms present in the air. In the absence of preservatives, these bacteria and other microorganisms will grow quickly in the container so it will need to be discarded. However, ISO Class 5, or better cleanrooms, contain much fewer bacteria than regular rooms. Strictly, opened or needle-punctured single-dose containers, such as, for example, bags, bottles, syringes, and vials of sterile products and compounded sterile preparations (CSPs) must be used within one (1) hour if air quality in the room where the container is opened is worse than ISO Class 5. If, however, the air quality in the room where the container is opened is equivalent to ISO Class 5 or better, the container must be discarded within 6 hours of opening. See, Table 1. In comparison, single-dose containers (or vials) exposed to ISO Class 5, or cleaner air, may be used up to 6 hours after initial needle puncture.

TABLE 1

| | | Particle Count | |
|---|---|---|---|
| Class Name | | | FS |
| ISO | U.S. FS | | 209E· |
| Class | 209E | ISO · m³ | ft³ |
| 3 | Class 1 | 35.2 | 1 |
| 4 | Class 10 | 352 | 10 |
| 5 | Class 100 | 3,520 | 100 |
| 6 | Class 1,000 | 35,200 | 1,000 |
| 7 | Class 10,000 | 352,000 | 10,000 |
| 8 | Class 100,000 | 3,520.000 | 100,000 |

The ISO Classification of Particulate Matter in Room Air, is adapted from former Federal Standard No. 209E, General Services Administration, Washington, D.C., 20407 (Sep. 11, 1992) and ISO 14644-1: 1999, Cleanrooms and associated controlled environments—Part 1: Classification of air cleanliness. For example, 3,520 particles of 0.5 m per m³ or larger (ISO Class 5) is equivalent to 100 particles per ft³ (Class 100) (1 m³=35.2 ft³). As depicted, limits are in particles of 0.5 m and larger per cubic meter [current ISO] and cubic feet [former Federal Standard No. 209E, FS 209E]).

Multiple-dose containers are formulated for removal of portions on multiple occasions because they usually contain antimicrobial preservatives. The BUD after initially entering or opening (for example, needle-punctured) multiple-dose containers is 28 days, unless otherwise specified by the manufacturer. See, for example, Chapter 51, entitled “Antimicrobial Effectiveness Testing” of the United States Pharmacopeia (USP).

Another aspect provides a parenteral Fc-containing protein preparation in an aqueous solution. The method includes including at least one type of Fc-containing protein, and a phenol or benzyl alcohol. In some aspects, the Fc-containing proteins may be stabilized by providing Fc-containing proteins in an aqueous solution containing phenol or benzyl alcohol, and filling a container with the Fc-containing proteins in the aqueous solution containing phenol or benzyl alcohol.

Further details of the disclosed methods and systems are provided below.

### A. Multi-Dose Container for a Parenteral Fc-Containing Protein Preparation

In one aspect, a multi-dose container for a Parenteral Fc-containing Protein Preparation is provided. For example, a multiple-dose container may include a parenteral Fc-containing protein preparation. The preparation may comprise, in an aqueous solution, at least one type of Fc-containing protein. The preparation may also comprise, in the aqueous solution, phenol or benzyl alcohol.

One or more of the following example features may be included. The Fc-containing protein may be at a concentration of about 0.1 mg/ml to about 500 mg/ml. Phenol may be at a concentration of about 1 mg/ml to about 10 mg/ml. Phenol may also be at a concentration of about 2 mg/ml to about 5 mg/ml. Phenol may further be at a concentration of about 3 mg/ml (about 0.3%), for example. Benzyl alcohol may be at a concentration of about 1 mg/ml to about 15 mg/ml. Benzyl alcohol may further be at a concentration of about 3 mg/ml to about 12 mg/ml. Benzyl alcohol may further be at a concentration of about 10 mg/ml (about 1%), for example. The container may have a capacity of 1 ml to 100 ml. The container also may have a capacity of 5 ml to 100 ml. The container may further have a capacity of 10 ml to 50 ml. The container may even further have a capacity of 20 ml to 40 ml. The container also may have a capacity of 30 ml. The Fc-containing protein may be a monoclonal antibody. The monoclonal antibody may be a bispecific antibody. The container may include two or more types of Fc-containing proteins. The Fc-containing protein may be a receptor Fc-fusion protein. The Fc-containing protein also may be a trap protein.

### B. Method of Preparing a Parenteral Fc-Containing Protein

The disclosed systems and methods can be used to prepare an parenteral Fc-containing protein in an aqueous solution. One aspect provides a parenteral Fc-containing protein preparation in an aqueous solution, at least one type of Fc-containing protein. The parenteral Fc-containing protein preparation may also include, in the aqueous solution, phenol and/or soluble phenol derivatives. Benzyl alcohol and/or soluble derivatives thereof also can be employed.

One or more of the following exemplary features may be included. The Fc-containing protein may be at a concentration of about 0.1 mg/ml to about 500 mg/ml. Phenol may be at a concentration of about 1 mg/ml to about 10 mg/ml. Phenol may also be at a concentration of about 2 mg/ml to about 5 mg/ml. Phenol may further be at a concentration of about 3 mg/ml (about 0.3%), for example. Benzyl alcohol may be at a concentration of about 1 mg/ml to about 15 mg/ml. Benzyl alcohol may further be at a concentration of about 3 mg/ml to about 12 mg/ml. Benzyl alcohol may further be at a concentration of about 10 mg/ml (about 1%), for example. The Fc-containing protein may be a monoclonal antibody. The monoclonal antibody may be a bispecific antibody. The preparation may include two or more types of Fc-containing proteins. The Fc-containing protein may be a receptor Fc-fusion protein. The Fc-containing protein also may be a trap protein.

### C. Method of Stabilizing an Fc-Containing Protein Preparation in an Aqueous Solution

In one aspect, the method for stabilizing an Fc-containing protein preparation in an aqueous solution are provided. For example, stabilizing an Fc-containing protein preparation may include the step of providing an Fc-containing protein in an aqueous solution containing phenol or benzyl alcohol. The method of stabilizing an Fc-containing protein preparation may also include the step of filling a container with the Fc-containing protein in an aqueous solution containing phenol or benzyl alcohol.

One or more of the following example features may be included. Phenol or benzyl alcohol may be added to an aqueous solution comprising the Fc-containing protein. The Fc-containing protein may be at a concentration of about 0.1 mg/ml to about 500 mg/ml. Phenol may be at a concentration of about 1 mg/ml to about 10 mg/ml. Phenol may also be at a concentration of about 2 mg/ml to about 5 mg/ml. Phenol may further be at a concentration of about 3 mg/ml (about 0.3%), for example. Benzyl alcohol may be at a concentration of about 1 mg/ml to about 15 mg/ml. Benzyl alcohol may further be at a concentration of about 3 mg/ml to about 12 mg/ml. Benzyl alcohol may further be at a concentration of about 10 mg/ml (about 1%), for example. The Fc-containing protein may be a monoclonal antibody. The monoclonal antibody may be a bispecific antibody. The preparation may include two or more types of Fc-containing proteins. The Fc-containing protein may be a receptor Fc-fusion protein. The Fc-containing protein also may be a trap protein.

### D. Proteins in the Protein Complexes

In one aspect, one of the proteins in the protein complex is a protein drug product or is a protein of interest suitable for expression in prokaryotic or eukaryotic cells. For example, the protein in the protein complexes can be an antibody or antigen-binding fragment thereof, a chimeric antibody or antigen-binding fragment thereof, an ScFv or fragment thereof, an Fc-fusion protein or fragment thereof, a growth factor or a fragment thereof, a cytokine or a fragment thereof, or an extracellular domain of a cell surface receptor or a fragment thereof. Proteins in the complexes may be simple polypeptides consisting of a single subunit, or complex multi-subunit proteins comprising two or more subunits. The protein of interest may be a biopharmaceutical product, food additive or preservative, or any protein product subject to purification and quality standards.

In some aspects, the protein in the protein complexes is an antibody, a human antibody, a humanized antibody, a chimeric antibody, a monoclonal antibody, a multi-specific antibody, a bispecific antibody, an antigen binding antibody fragment, a single chain antibody, a diabody, triabody or tetrabody, a dual-specific, tetravalent immunoglobulin G-like molecule, termed dual variable domain immunoglobulin (DVD-IG), an IgD antibody, an IgE antibody, an IgM antibody, an IgG antibody, an IgG1 antibody, an IgG2 antibody, an IgG3 antibody, or an IgG4 antibody. In one aspect, the antibody is an IgG1 antibody. In one aspect, the antibody is an IgG2 antibody. In one aspect, the antibody is an IgG4 antibody. In another aspect, the antibody comprises a chimeric hinge. In still other aspects, the antibody comprises a chimeric Fc. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody.

In some aspects, the antibody is selected from the group consisting of an anti-Programmed Cell Death 1 antibody (for example, an anti-PD1 antibody as described in U.S. Pat. Appln. Pub. No. US2015/0203579A1), an anti-Programmed Cell Death Ligand-1 (for example, an anti-PD-L1 antibody as described in in U.S. Pat. Appln. Pub. No. US2015/0203580A1), an anti-D114 antibody, an anti-Angiopoetin-2 antibody (for example, an anti-ANG2 antibody as described in U.S. Pat. No. 9,402,898), an anti-Angiopoetin-Like 3 antibody (for example, an anti-AngPtl3 antibody as described in U.S. Pat. No. 9,018,356), an anti-platelet derived growth factor receptor antibody (for example, an anti-PDGFR antibody as described in U.S. Pat. No. 9,265,827), an anti-Erb3 antibody, an anti-Prolactin Receptor antibody (for example, anti-PRLR antibody as described in U.S. Pat. No. 9,302,015), an anti-Complement 5 antibody (for example, an anti-C5 antibody as described in U.S. Pat. Appln. Pub. No US2015/0313194A1), an anti-TNF antibody, an anti-epidermal growth factor receptor antibody (for example, an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (for example, an anti-PCSK9 antibody as described in U.S. Pat. No. 8,062,640 or U.S. Pat. No. 9,540,449), an Anti-Growth and Differentiation Factor-8 antibody (for example an anti-GDF8 antibody, also known as anti-myostatin antibody, as described in U.S. Pat Nos. 8,871,209 or 9,260,515), an anti-Glucagon Receptor (for example anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (for example, an anti-IL4R antibody as described in U.S. Pat. Appln. Pub. No. US2014/0271681A1 or U.S. Pat Nos. 8,735,095 or 8,945,559), an anti-interleukin 6 receptor antibody (for example, an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (for example, anti-IL33 antibody as described in U.S. Pat. Nos. 9,453,072 or 9,637,535), an anti-Respiratory syncytial virus antibody (for example, anti-RSV antibody as described in U.S. Pat. Appln. Pub. No. 9,447,173), an anti-Cluster of differentiation 3 (for example, an anti-CD3 antibody, as described in U.S. Pat. Nos. 9,447,173 and 9,447,173, and in U.S. Application No. 62/222,605), an anti-Cluster of differentiation 20 (for example, an anti-CD20 antibody as described in U.S. Pat. No. 9,657,102 and US20150266966A1, and in U.S. Pat. No. 7,879,984), an anti-CD19 antibody, an anti-CD28 antibody, an anti-Cluster of Differentiation-48 (for example anti-CD48 antibody as described in U.S. Pat. No. 9,228,014), an anti-Fel d1 antibody (for example as described in U.S. Pat. No. 9,079,948), an anti-Middle East Respiratory Syndrome virus (for example an anti-MERS antibody as described in U.S. Pat. Appln. Pub. No. US2015/0337029A1), an anti-Ebola virus antibody (for example as described in U.S. Pat. Appln. Pub. No. US2016/0215040), an anti-Zika virus antibody, an anti-Lymphocyte Activation Gene 3 antibody (for example an anti-LAG3 antibody, or an anti-CD223 antibody), an anti-Nerve Growth Factor antibody (for example an anti-NGF antibody as described in U.S. Pat. Appln. Pub. No. US2016/0017029 and U.S. Pat. Nos. 8,309,088 and 9,353,176) and an anti-Protein Y antibody. In some aspects, the bispecific antibody is selected from the group consisting of an anti-CD3×anti-CD20 bispecific antibody (as described in U.S. Pat. Appln. Pub. Nos. US2014/0088295A1 and US20150266966A1), an anti-CD3×anti-Mucin 16 bispecific antibody (for example, an anti-CD3×anti-Mucl6 bispecific antibody), and an anti-CD3×anti-Prostate-specific membrane antigen bispecific antibody (for example, an anti-CD3×anti-PSMA bispecific antibody). In some aspects, the protein of interest is selected from the group consisting of abciximab, adalimumab, adalimumab-atto, ado-trastuzumab, alemtuzumab, alirocumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cemiplimab, cetuximab, denosumab, dinutuximab, dupilumab, durvalumab, eculizumab, elotuzumab, emicizumab-kxwh, emtansinealirocumab, evinacumab, evolocumab, fasinumab, golimumab, guselkumab, ibritumomab tiuxetan, idarucizumab, infliximab, infliximab-abda, infliximab-dyyb, ipilimumab, ixekizumab, mepolizumab, necitumumab, nesvacumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, sarilumab, secukinumab, siltuximab, tocilizumab, tocilizumab, trastuzumab, trevogrumab, ustekinumab, and vedolizumab.

In some aspects, the protein in the complexes is a recombinant protein that contains an Fc moiety and another domain, (for example, an Fc-fusion protein). In some aspects, an Fc-fusion protein is a receptor Fc-fusion protein, which contains one or more extracellular domain(s) of a receptor coupled to an Fc moiety. In some aspects, the Fc moiety comprises a hinge region followed by a CH2 and CH3 domain of an IgG. In some aspects, the receptor Fc-fusion protein contains two or more distinct receptor chains that bind to either a single ligand or multiple ligands. For example, an Fc-fusion protein is a TRAP protein, such as for example an IL-1 Trap (for example, rilonacept, which contains the IL-1RAcP ligand binding region fused to the Il-1R1 extracellular region fused to Fc of hIgG1; See, U.S. Pat. No. 6,927,044, which is herein incorporated by reference in its entirety), or a VEGF Trap (for example, aflibercept or ziv-aflibercept, which comprises the Ig domain 2 of the VEGF receptor Fltl fused to the Ig domain 3 of the VEGF receptor Flkl fused to Fc of hIgGl; See, U.S. Pat. Nos. 7,087,411 and 7,279,159). In other aspects, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

### EXAMPLES

The inventions are further described by the following examples, which are illustrative of the many aspects of the inventions, but do not limit the inventions in any manner.

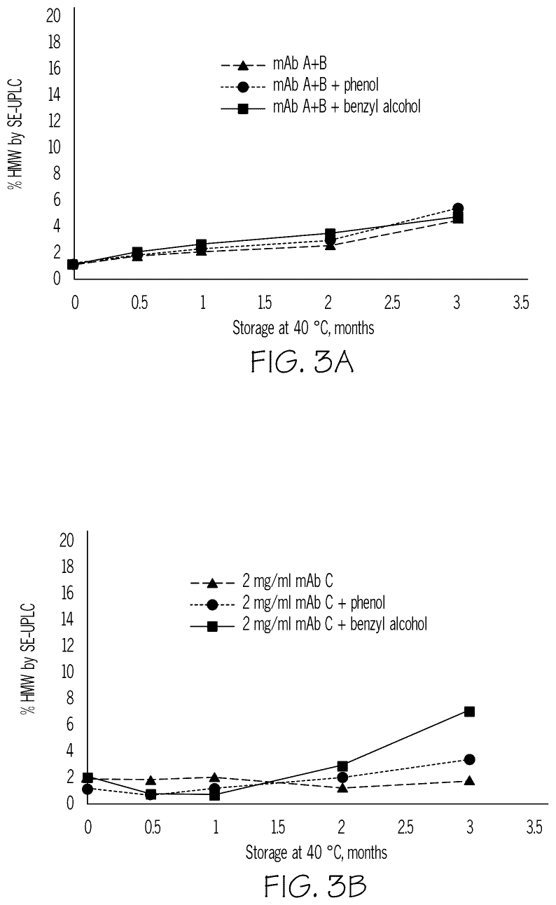

The impact of typical antimicrobial preservatives on stability of the liquid formulations of several mAbs were assessed by including preservatives into five formulated drug substance (FDS) formulations with stability of the resulting formulations assessed at different temperatures over time. The compatibility of chlorobutanol, m-cresol, phenol and benzyl alcohol were assessed with the several mAb formulations. It was found that chlorobutanol and m-cresol were not compatible with the assessed formulations so only phenol and benzyl alcohol were tested further. Long-term stability of preservative-spiked formulations were tested in a variety of conditions, including at various temperatures and relative humidity (RH) (for example, at 5° C., 25° C./60% RH, 40° C./75% RH and at 45° C.). The critical quality attributes tested included solution clarity by UV absorbance at 405 nm, subvisible particle formation by MFI, aggregate formation using SE-UPLC, protein concentration using RP-UPLC and protein charge variants using CEX. Compendial assays were used to evaluate antimicrobial effectiveness of preservatives. See, for example, Chapter 51 of the United States Pharmacopeia (USP). Stability of all mAb formulations in the presence of preservatives was assessed at 5° C. and 25° C. for up to 24 months as determined by the methods listed below. At the stress conditions (for example, at 40° C. and 45° C.), however, preservatives increased mAb aggregation as detected by SE-UPLC in some of the formulations (for example, FIGS. 4A-4D). Effect of preservatives on mAb aggregation depended on the nature of mAb, preservative and mAb concentration. Preservatives did not influence protein charge or subvisible particle formation even in stress conditions. Effectiveness of the preservatives in reducing microbial activity in these formulations was also evaluated.

### Example 1: Effect of Preservatives on mAbs Formulation Turbidity Under the Same Stress Conditions

### Methods

### Formulation Turbidity Under the Same Stress Condition

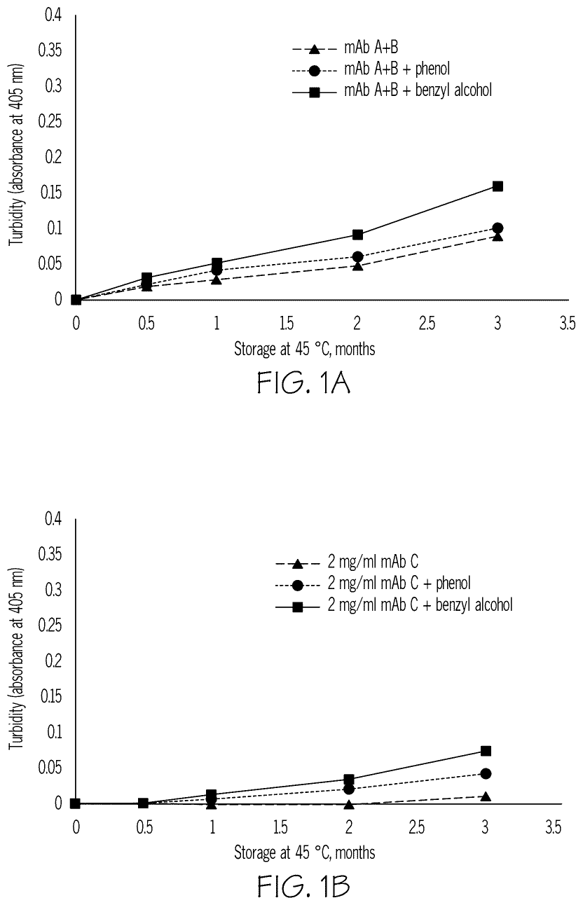

To determine formulation turbidity for different mAbs formulations, various antibodies were incubated at 45° C. over the course of 3 months with or without tested preservatives. In a first phase, the mAbs formulation included 120 mg/mL of mAb A+B stored at 45° C. without preservatives, 120 mg/mL of mAb A+B with phenol, and 120 mg/mL of mAb A+B with benzyl alcohol, respectively. See, FIG. 1A. In a second phase, the mAbs formulation included 2 mg/mL of mAb C stored at 45° C. without preservatives, 2 mg/mL of mAb C with phenol, and 2 mg/mL of mAb C with benzyl alcohol, respectively. See, FIG. 1B. In a third phase, the mAbs formulation included 200 mg/mL of mAb D stored at 45° C. without preservatives, 200 mg/mL of mAb D with phenol, and 200 mg/mL of mAb D with benzyl alcohol, respectively. See, FIG. 1C. In a fourth phase, the mAbs formulation included 100 mg/mL of mAb C stored at 45° C. without preservatives, 100 mg/mL of mAb C with phenol, and 100 mg/mL of mAb C with benzyl alcohol, respectively. See, FIG. 1D.

### Analysis of Formulation Turbidity Under the Same Stress Condition

At the end of 3 months, each of the four different mAbs formulations including the preservative benzyl alcohol had a relatively higher formulation turbidity when incubated at 45° C. than when the mAbs formulation was combined with the preservative phenol or with no preservative at all. Overall, the mAbs formulation including 2 mg/mL of mAb C with benzyl alcohol showed the least increase of turbidity, and the mAbs formulation including 200 mg/mL of mAb D with benzyl alcohol showed the highest increase in turbidity.

### Example 2: Effect of Preservatives on mAbs Formulation Turbidity Under the Different Stress Conditions

### Methods

### Formulation Turbidity Under Different Stress Conditions

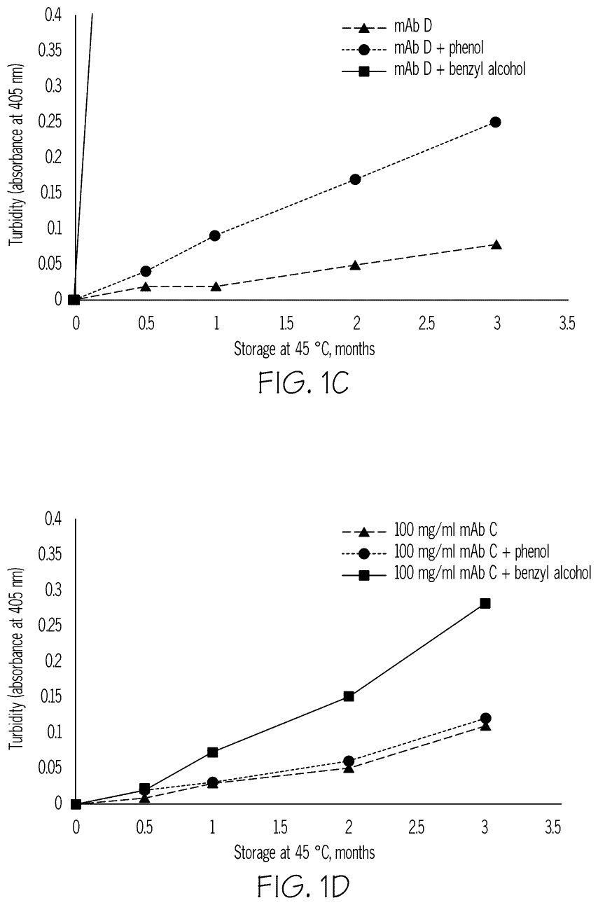

To determine formulation turbidity for different mAbs formulations, various antibodies were incubated at 25° C. over the course of 6 months with or without preservatives, and at 5° C. over the course of 24 months with or without the same tested preservatives. In a first phase where the antibodies were incubated at 25° C. over the course of 6 months, the mAbs formulations included: 2 mg/mL of mAb C without preservatives, 2 mg/mL of mAb C with phenol, and 2 mg/mL of mAb C with benzyl alcohol, respectively; 100 mg/mL of mAb C without preservatives, 100 mg/mL of mAb C with phenol, and 100 mg/mL of mAb C with benzyl alcohol, respectively; 120 mg/mL of mAb A+B without preservatives, 120 mg/mL of mAb A+B with phenol, and 120 mg/mL of mAb A+B with benzyl alcohol, respectively; and 200 mg/mL of mAb D without preservatives, 200 mg/mL of mAb D with phenol, and 200 mg/mL of mAb D with benzyl alcohol, respectively. See, FIG. 2A. In a second phase where the antibodies were incubated at 5° C. over the course of 24 months, the mAbs formulations included: 2 mg/mL of mAb C without preservatives, 2 mg/mL of mAb C with phenol, and 2 mg/mL of mAb C with benzyl alcohol, respectively; 100 mg/mL of mAb C without preservatives, 100 mg/mL of mAb C with phenol, and 100 mg/mL of mAb C with benzyl alcohol, respectively; 120 mg/mL of mAb A+B without preservatives, 120 mg/mL of mAb A+B with phenol, and 120 mg/mL of mAb A+B with benzyl alcohol, respectively; and 200 mg/mL of mAb D without preservatives, 200 mg/mL of mAb D with phenol, and 200 mg/mL of mAb D with benzyl alcohol, respectively. See, FIG. 2B.

### Analysis of Formulation Turbidity Under Different Stress Conditions

There was no appreciable instability observed when the formulations were incubated at 25° C. for 6 months or at 5° C. for 24 months with the tested preservatives.

### Example 3: Effect of Preservatives on High Molecular Weight (HMW) Complex Formation when Stored at 40° C. for 3 Months

### Methods

HMW Complex Formation when stored at 40° C.

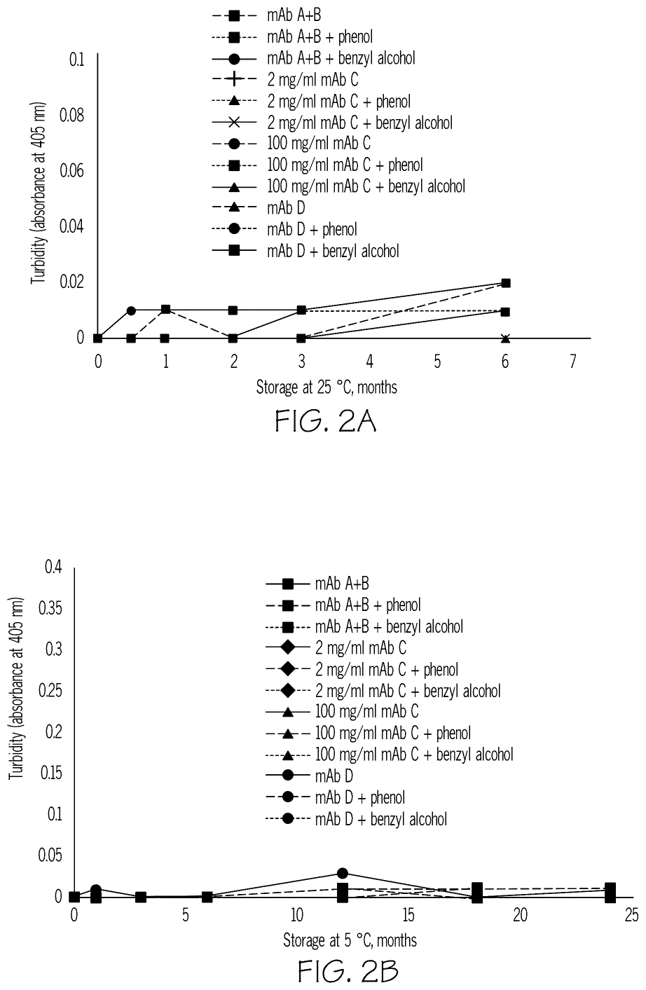

To determine the effect of preservatives on high molecular weight (HMW) complex formation for different mAbs formulations, various antibodies were incubated at 40° C. over the course of 3 months with or without tested preservatives. In a first phase, the mAbs formulation included 120 mg/mL of mAb A+B stored at 40° C. without preservatives, 120 mg/mL of mAb A+B with phenol, and 120 mg/mL of mAb A+B with benzyl alcohol, respectively. See, FIG. 3A. In a second phase, the mAbs formulation included 2 mg/mL of mAb C stored at 40° C. without preservatives, 2 mg/mL of mAb C with phenol, and 2 mg/mL of mAb C with benzyl alcohol, respectively. See, FIG. 3B. In a third phase, the mAbs formulation included 200 mg/mL of mAb D stored at 40° C. without preservatives, with 200 mg/mL of mAb D phenol, and 200 mg/mL of mAb D with benzyl alcohol, respectively. See, FIG. 3C. In a fourth phase, the mAbs formulation included 100 mg/mL of mAb C stored at 40° C. without preservatives, 100 mg/mL of mAb C with phenol, and 100 mg/mL of mAb C with benzyl alcohol, respectively. See, FIG. 3D.

Analysis of HMW Complex Formation when Stored at 40° C.

At the end of 3 months, each of the four different mAbs formulations including the preservative benzyl alcohol had a relatively higher destabilization effect when incubated at 40° C. than when the mAbs formulation was combined with the preservative phenol or with no preservative at all. Overall, the mAbs formulation including 120 mg/mL of mAb A+B showed the least destabilization effect, if at all, and the mAbs formulation including 200 mg/mL of mAb D with benzyl alcohol showed the most destabilization effect.

### Example 4: Effect of Preservatives on High Molecular Weight (HMW) Complex Formation when Stored at 25° C. for 6 Months

### Methods

HMW Complex Formation when Stored at 25° C.

To determine the effect of preservatives on high molecular weight (HMW) complex formation for different mAbs formulations, various antibodies were incubated at 25° C. over the course of 6 months with or without tested preservatives. In a first phase, the mAbs formulation included 120 mg/mL of mAb A+B stored at 25° C. without preservatives, 120 mg/mL of mAb A+B with phenol, and 120 mg/mL of mAb A+B with benzyl alcohol, respectively. See, FIG. 4A. In a second phase, the mAbs formulation included 2 mg/mL of mAb C stored at 25° C. without preservatives, 2 mg/mL of mAb C with phenol, and 2 mg/mL of mAb C with benzyl alcohol, respectively. See, FIG. 4B. In a third phase, the mAbs formulation included 200 mg/mL of mAb D stored at 25° C. without preservatives, 200 mg/mL of mAb D with phenol, and 200 mg/mL of mAb D with benzyl alcohol, respectively. See, FIG. 4C. In a fourth phase, the mAbs formulation included 100 mg/mL of mAb C stored at 25° C. without preservatives, 100 mg/mL of mAb C with phenol, and 100 mg/mL of mAb C with benzyl alcohol, respectively. See, FIG. 4D.

Analysis of HMW Complex Formation when Stored at 25° C.

At the end of 6 months, there was no appreciable destabilization effect on the percent of high molecular weight (HMW) complex formation from added preservatives when incubated at 25° C.

### Example 5: Effect on the Stability of Antibodies with Preservatives when Stored at 5° C. for 24 Months

### Methods

Stability of Antibodies when Stored with Preservatives at 5° C.

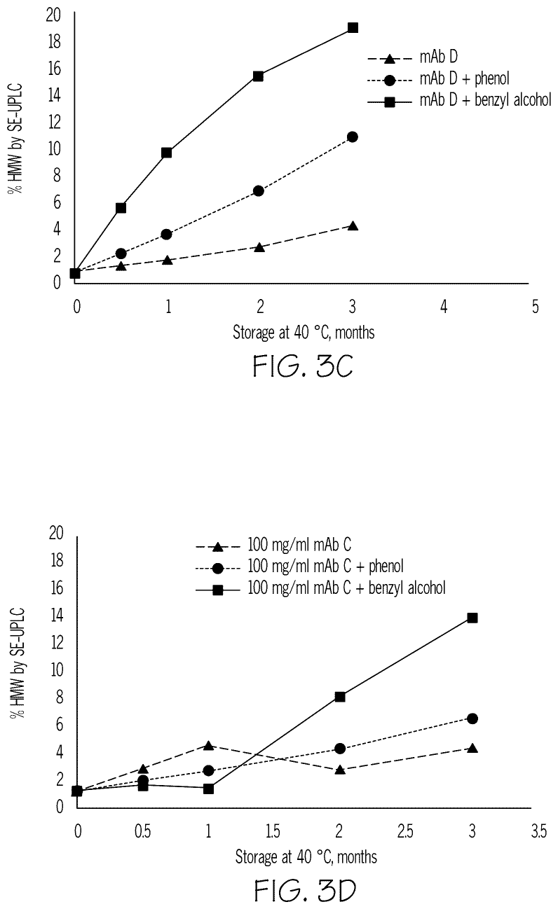

To determine the stability of different mAbs formulations, various antibodies were incubated at 5° C. over the course of 24 months with or without a tested preservative. The mAbs formulations included: 2 mg/mL of mAb C without preservatives, 2 mg/mL of mAb C with phenol, and 2 mg/mL of mAb C with benzyl alcohol, respectively; 100 mg/mL of mAb C without preservatives, 100 mg/mL of mAb C with phenol, and 100 mg/mL of mAb C with benzyl alcohol, respectively; 120 mg/mL of mAb A+B without preservatives, 120 mg/mL of mAb A+B with phenol, and 120 mg/mL of mAb A+B with benzyl alcohol, respectively; and 200 mg/mL of mAb D without preservatives, 200 mg/mL of mAb D with phenol, and 200 mg/mL of mAb D with benzyl alcohol, respectively. See, FIG. 5.

Analysis of the Stability of Antibodies when Stored with Preservatives at 5° C.

At the end of 24 months, there was no appreciable instability observed when the mAbs formulations (drug products) were incubated at 5° C. with tested preservatives.

### Example 6: Impact on Charge Variant Formation in Antibodies with Preservatives Incubated at 40° C.

### Methods

Charge Variant Formation of Antibodies with Preservatives when Stored at 40° C.

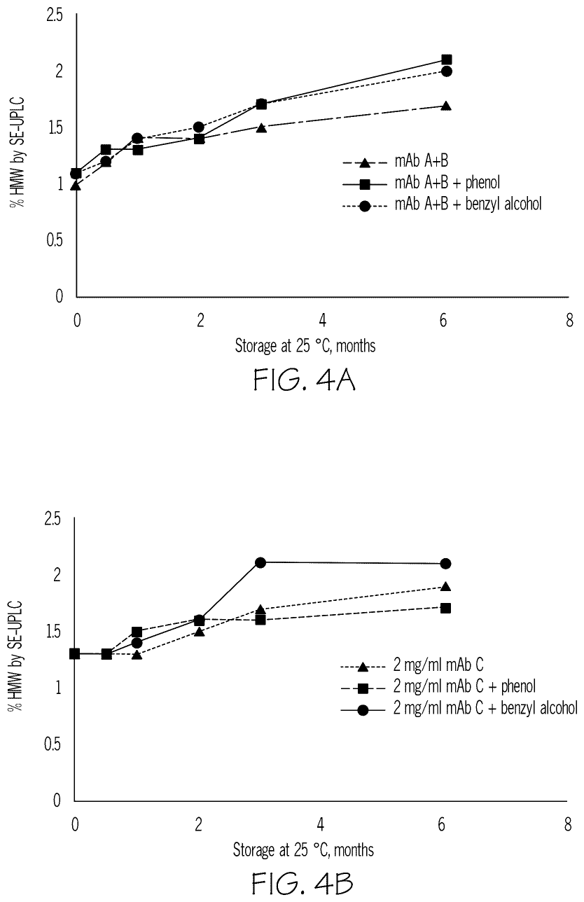

To determine the impact on the charge variant formation for different mAbs formulations, various antibodies were incubated at 40° C. over the course of 2 months with and without tested preservatives. In a first phase, the mAbs formulation included 2 mg/mL of mAb C stored at 40° C. without preservatives, 2 mg/mL of mAb C with phenol, and 2 mg/mL of mAb C with benzyl alcohol, respectively. See, FIG. 6A. In a second phase, the mAbs formulation included 200 mg/mL of mAb D stored at 40° C. without preservatives, 200 mg/mL of mAb D with phenol, and 200 mg/mL of mAb D with benzyl alcohol, respectively. See, FIG. 6B. In a third phase, the mAbs formulation included 100 mg/mL of mAb C stored at 40° C. without preservatives, 100 mg/mL of mAb C with phenol, and 100 mg/mL of mAb C with benzyl alcohol, respectively. See, FIG. 6C.

Analysis of Charge Variant Formation of Antibodies with Preservatives when Stored at 40° C.

At the end of 2 months, there was no substantial destabilization effect observed for antibody charge variants from preservatives when the mAbs formulations were incubated at 40° C.

### Example 7: Impact on Charge Variant Formation in Antibodies with Preservatives Incubated at 25° C.

### Methods

Charge Variant Formation of Antibodies with Preservatives when Stored at 25° C.

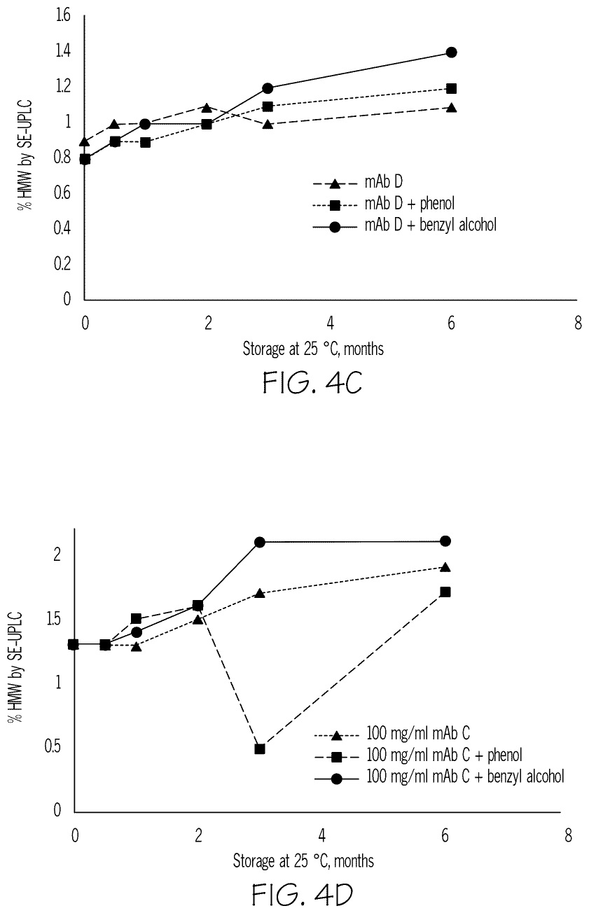

To determine the impact on the charge variant formation for different mAbs formulations, various antibodies were incubated at 25° C. over the course of 6 months with tested preservatives. In a first phase, the mAbs formulation included 2 mg/mL of mAb C stored at 25° C. without preservatives, 2 mg/mL of mAb C with phenol, and 2 mg/mL of mAb C with benzyl alcohol, respectively. See, FIG. 7A. In a second phase, the mAbs formulation included 200 mg/mL of mAb D stored at 25° C. without preservatives, 200 mg/mL of mAb D with phenol, and 200 mg/mL of mAb D with benzyl alcohol, respectively. See, FIG. 7B. In a third phase, the mAbs formulation included 100 mg/mL of mAb C stored at 25° C. without preservatives, with 100 mg/mL of mAb C phenol, and 100 mg/mL of mAb C with benzyl alcohol, respectively. See, FIG. 7C.

Analysis of Charge Variant Formation of Antibodies with Preservatives when Stored at 25° C.

At the end of 6 months, there was no substantial instability observed for antibody charge variants when the mAbs formulations were incubated at 25° C. with tested preservatives.

### Example 8: Impact on Charge Variant Formation in Antibodies with Preservatives Incubated at 5° C.

### Methods

Impact on Charge Variant Formation for Different Antibody Formulations with Preservatives when Stored at 5° C. for 12 Months and 24 Months.

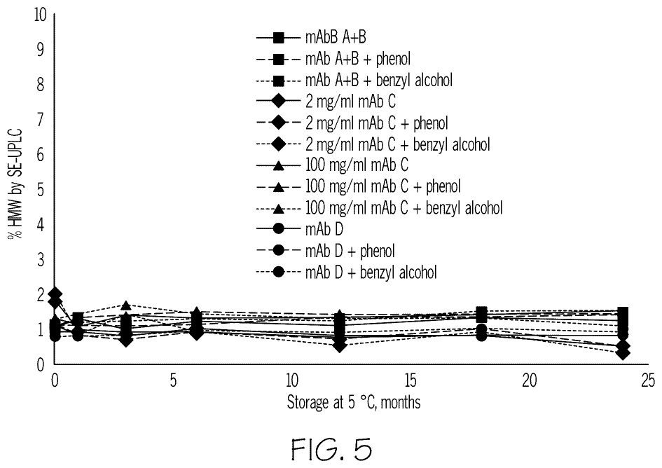

Various antibodies were incubated at 5° C. and over the course of 12 months with or without tested preservatives. In a first phase, the mAbs formulation included 200 mg/mL of mAb D stored at 5° C. without preservatives, 200 mg/mL of mAb D with phenol, and 200 mg/mL of mAb D with benzyl alcohol, respectively. See, FIG. 8A.

Various antibodies were also incubated at 5° C. and over the course of 24 months with or without tested preservatives. In a first phase, the mAbs formulation included 200 mg/mL of mAb D stored at 5° C. without preservatives, 200 mg/mL of mAb D with phenol, and 200 mg/mL of mAb D with benzyl alcohol, respectively. In a second phase, the mAbs formulation included 2 mg/mL of mAb C stored at 5° C. without preservatives, 2 mg/mL of mAb C with phenol, and 2 mg/mL of mAb C with benzyl alcohol, respectively; and 100 mg/mL of mAb C stored at 5° C. without preservatives, 100 mg/mL of mAb C with phenol, and 100 mg/mL of mAb C with benzyl alcohol, respectively. See, FIG. 8B.

Analysis of Charge Variant Formation of Antibodies with Preservatives when Stored at 25° C.

At the end of both 12 and 24 month incubation periods, there was no appreciable instability observed for antibody charge variants when the mAbs formulations were incubated at 5° C. with tested preservatives.

### Example 9: Effect on Subvisible Particle Formation in mAbs Formulations with or without Preservatives Incubated at 5° C. for 24 Months, 25° C. for 6 Months, and 40° C. for 3 Months

### Methods

Subvisible Particle Formation in mAbs Formulations when Incubated at 5° C., 25° C., and 40° C.

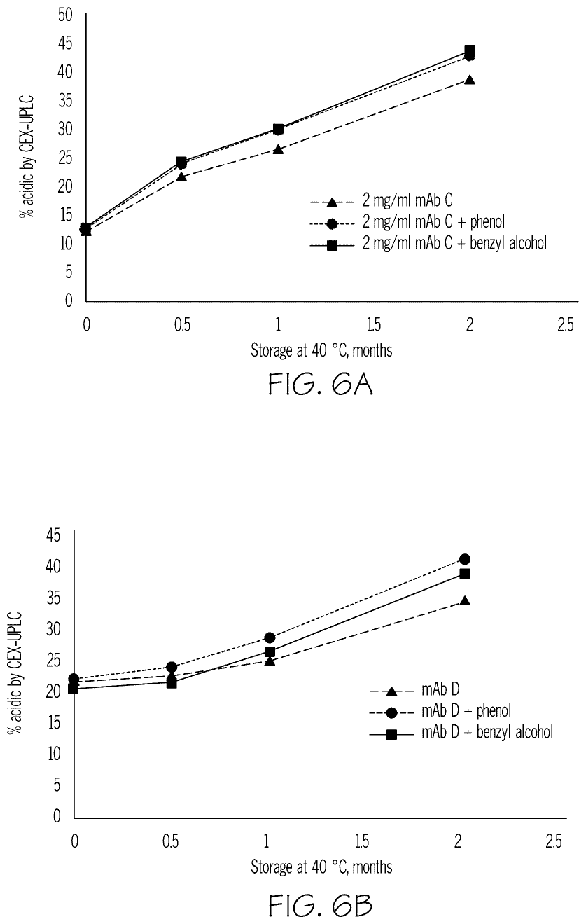

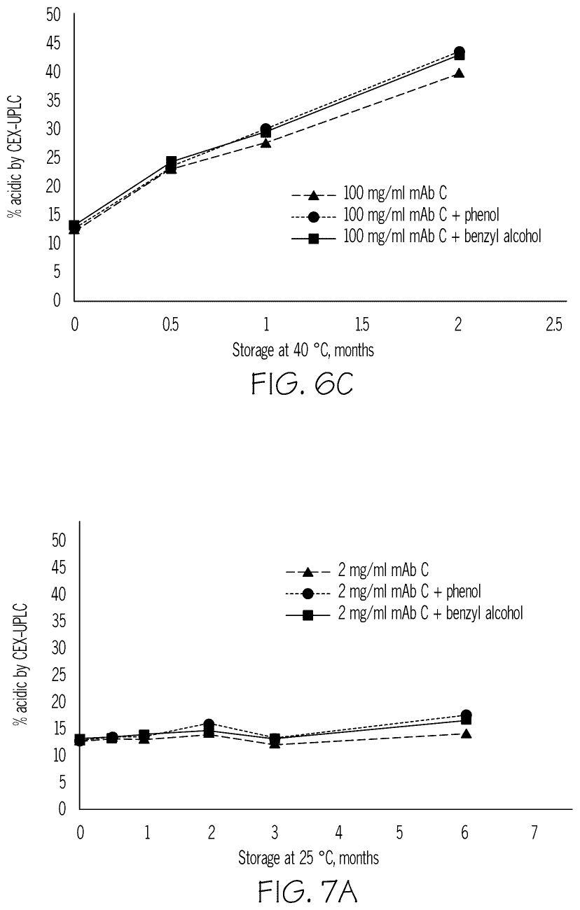

To determine the impact on the charge variant formation for different mAbs formulations, various antibodies were incubated, with or without tested preservatives, at 5° C. over the course of 24 months, 25° C. over the course of 6 months, and 40° C. over the course of 3 months. In a first phase where the antibodies were incubated at 5° C. over the course of 24 months, the mAbs formulations included: 2 mg/mL of mAb C without preservatives, 2 mg/mL of mAb C with phenol, and 2 mg/mL of mAb C with benzyl alcohol, respectively; 100 mg/mL of mAb C without preservatives, 100 mg/mL of mAb C with phenol, and 100 mg/mL of mAb C with benzyl alcohol, respectively; 120 mg/mL of mAb A+B without preservatives, 120 mg/mL of mAb A+B with phenol, and 120 mg/mL of mAb A+B with benzyl alcohol, respectively; and 200 mg/mL of mAb D without preservatives, 200 mg/mL of mAb D with phenol, and 200 mg/mL of mAb D with benzyl alcohol, respectively. See, FIG. 9A.

In a second phase where the antibodies were incubated at 25° C. over the course of 6 months, the mAbs formulations included: 2 mg/mL of mAb C without preservatives, 2 mg/mL of mAb C with phenol, and 2 mg/mL of mAb C with benzyl alcohol, respectively; 100 mg/mL of mAb C without preservatives, 100 mg/mL of mAb C with phenol, and 100 mg/mL of mAb C with benzyl alcohol, respectively; 120 mg/mL of mAb A+B without preservatives, 120 mg/mL of mAb A+B with phenol, and 120 mg/mL of mAb A+B with benzyl alcohol, respectively; and 200 mg/mL of mAb D without preservatives, 200 mg/mL of mAb D with phenol, and 200 mg/mL of mAb D with benzyl alcohol, respectively. See, FIG. 9B.

In a third phase where the antibodies were incubated at 40° C. over the course of 3 months, the mAbs formulations included: 2 mg/mL of mAb C without preservatives, 2 mg/mL of mAb C with phenol, and 2 mg/mL of mAb C with benzyl alcohol, respectively; 100 mg/mL of mAb C without preservatives, 100 mg/mL of mAb C with phenol, and 100 mg/mL of mAb C with benzyl alcohol, respectively; 120 mg/mL of mAb A+B without preservatives, 120 mg/mL of mAb A+B with phenol, and 120 mg/mL of mAb A+B with benzyl alcohol, respectively; and 200 mg/mL of mAb D without preservatives, 200 mg/mL of mAb D with phenol, and 200 mg/mL of mAb D with benzyl alcohol, respectively. See, FIG. 9C.

Analysis of Subvisible Particle Formation in mAbs Formulations when Incubated at 5° C., 25° C., and 40° C.

There was no subvisible formation observed for the mAbs formulations with or without tested preservatives when incubated at 5° C., 25° C., and 40° C.

Overall, there was no appreciable destabilizing effect on the mAbs formulations when incubated at 5° C. for 24 months, with or without the tested preservatives, and phenol was indicated to create the least destabilization effect, thereby presenting as the more promising preservative for use in stabilizing mAbs formulations. Out of 4 common preservatives tested, cresol and chlorobutanol were shown to be incompatible with common mAb formulations as the caused turbidity. Benzyl alcohol under a stressed condition, which demonstrated degradation pathways, showed higher mAb aggregation as evidenced by HMW species detected by SE-UPLC, turbidity and subvisible particle count. Phenol and benzyl alcohol showed only minimal destabilizing effect on mAbs.

It is concluded that preservatives may destabilize antibodies especially for high concentration protein formulations at high temperatures, particularly as applied to formulation turbidity and the percentage of high molecular weight formation. Both phenol and benzyl alcohol may be used as potential preservatives in antibody formulations (such as, mAb liquid formulations). Preservative effect in stress conditions depends on preservative identity, mAb identity and mAb concentration. Preservatives did not influence protein charge or subvisible particle formation even in stress conditions. As such, mAb stability at stress conditions needs to be evaluated for each individual formulation prior to the preservative selection.

### Example 10: Testing Antibacterial Antibody Formulations Comprising Phenol or Benzyl Alcohol

### Methods

Antibacterial antibody formulations comprising phenol or benzyl alcohol were tested against Escherichia Coli or Staphylococcus Aureus. See, Table 2 below.

TABLE 2

| | | | Sample |
|---|---|---|---|
| | | | Incubation |
| Organism | ATCC # | Selection Rational | Temperature |
| Escherichia coli (Ec) | 8739 | Gram-negative, | 20-25° C. |
| | | USP<51> Organism | |
| Staphylococcus | 6538 | Gram-positive, | 20-25° C. |
| aureus (Sa) | | USP<51> Organism | |

### Method Qualification

For each formulation containing preservative (phenol or benzyl alcohol), 2 ml was removed from the stock and 1 ml was transferred into two separate tubes containing 10 mL of Tryptic Soy Broth (TSB). The tubes were then inoculated with 1×10⁶-1×10⁷ CFU/mL of one of the two organisms. Each tube was vortexed for 10 seconds to ensure homogeneity before 1 ml was removed from the tube and plated in duplicate onto empty petri dishes. The same procedure was repeated with phosphate buffered saline (PBS) instead of the formulation. Melted and tempered Tryptic Soy Agar (TSA) was then poured over the samples and gently swirled. The agar was allowed to solidify, and the plates were placed in the 30-35° C. incubator.

The plates were counted after 5 days of incubation and averaged together for the final CFU count. The total CFU was compared between the PBS controls and the formulation samples with an acceptable recovery being between 50-200% of the control.

### Inoculation and Incubation

10 mL of each formulation of drug product was transferred into separate 10 mL Falcon tubes and were inoculated with each challenge organism to a target final countable concentration of 1×10⁶-1×10⁷ CFU/mL. Following inoculation, the 50 mL tubes were stored at 20-25° C. for up to 28 days.

### Sampling

To evaluate microbial growth, samples were taken at designated timepoints and tested for bioburden determination using serial dilution required for sample analysis. At each timepoint for Escherichia coli, prior to sampling, the 50 ml conical tubes were vortexed for at least 10 seconds. Using a 200 ul pipette, 100 ul was withdrawn from each tube and diluted into 10 ml of PBS. The dilution was vortexed for an additional 10 seconds, before 3 ml was withdrawn from the tube. 1 ml was diluted further into an additional 9 mL of phosphate buffered saline and 1 ml was plated in duplicate in empty petri dishes. The second dilution was vortexed for an additional 10 seconds, 2 ml was subsequently removed and 1 ml was plated in duplicate in empty petri dishes.

For Staphylococcus aureus, the samples for all formulations were diluted to 1:1,000 and 1:10,000 for the first timepoint. For all other timepoints, the samples were diluted in the same manner as described for Escherichia coli. Tempered TSA was poured over the samples and allowed to solidify on the benchtop before being placed in the correct incubator based on microorganism. Plates were counted after 5 days of incubation. Table 3 summarizes the detail of sampling timepoints at the corresponding temperatures.

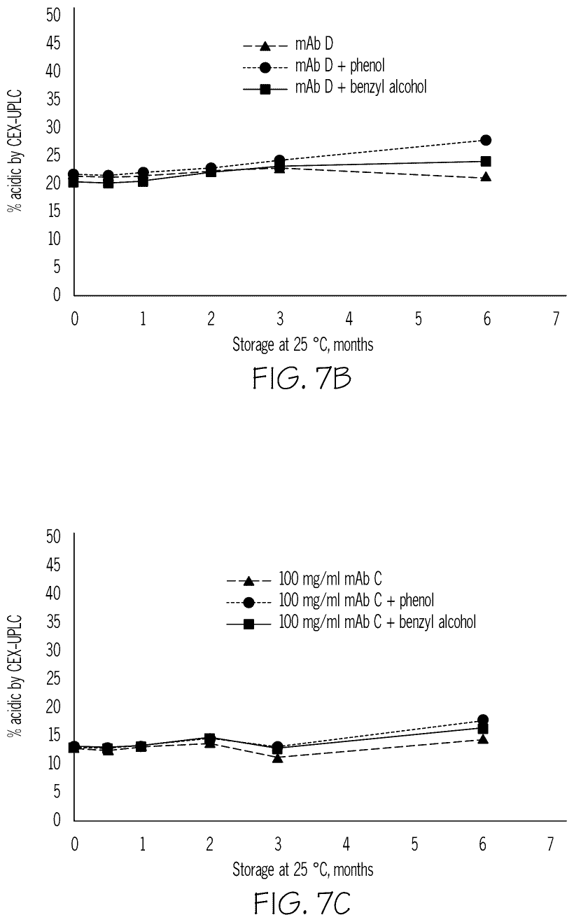

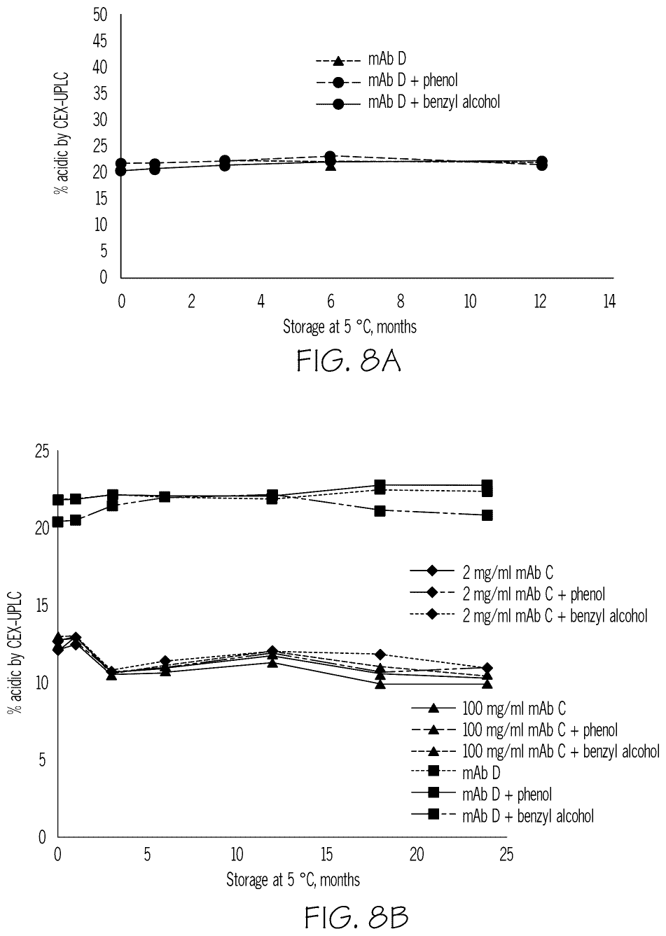

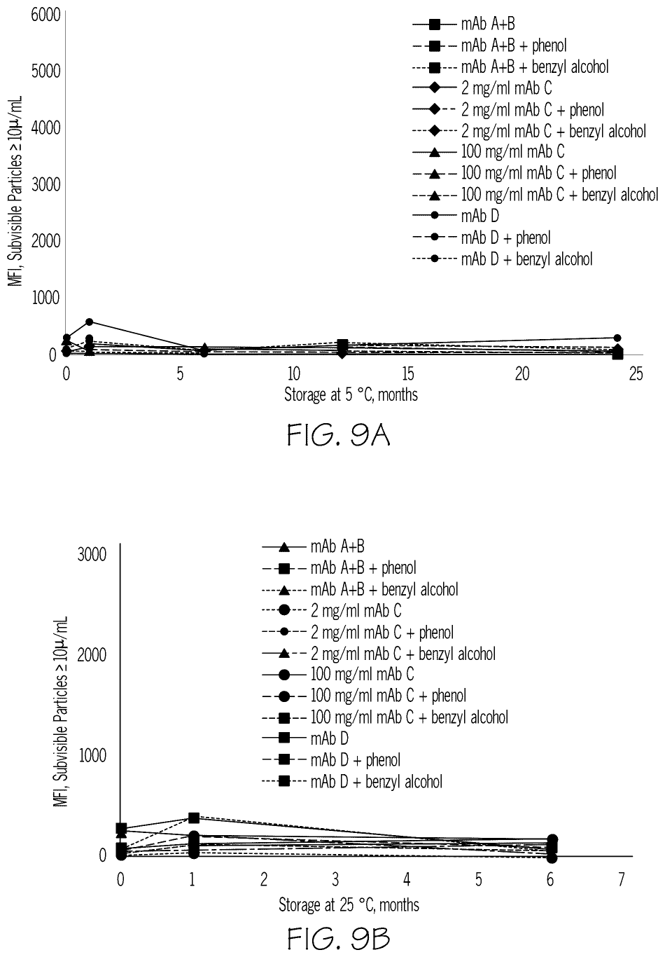

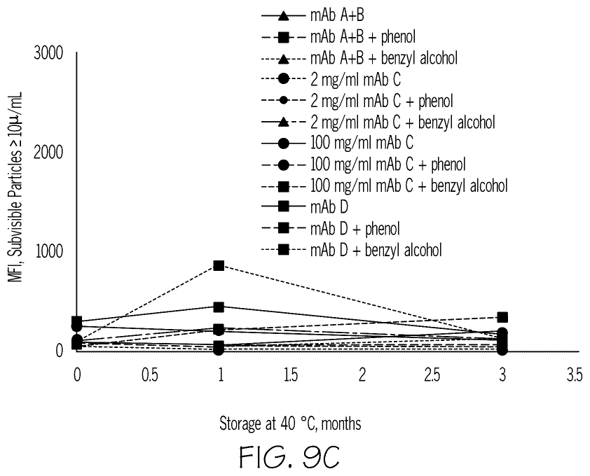

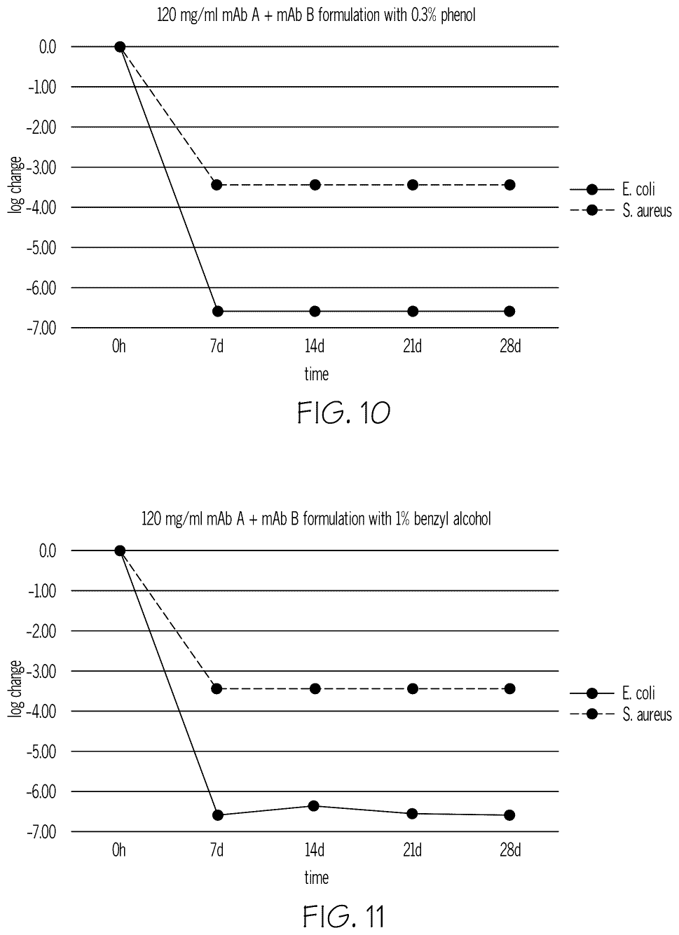

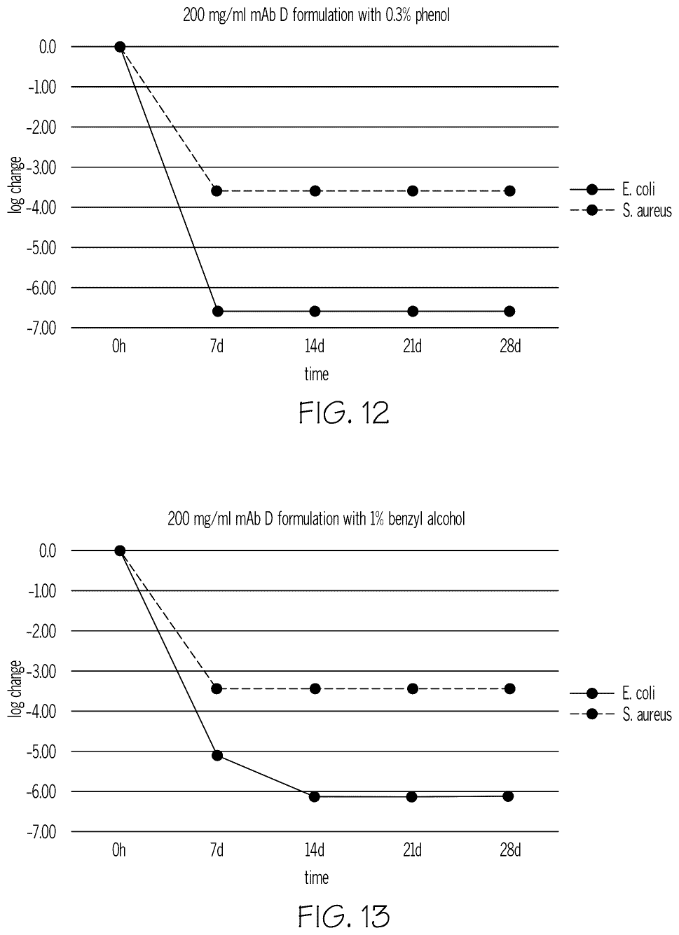

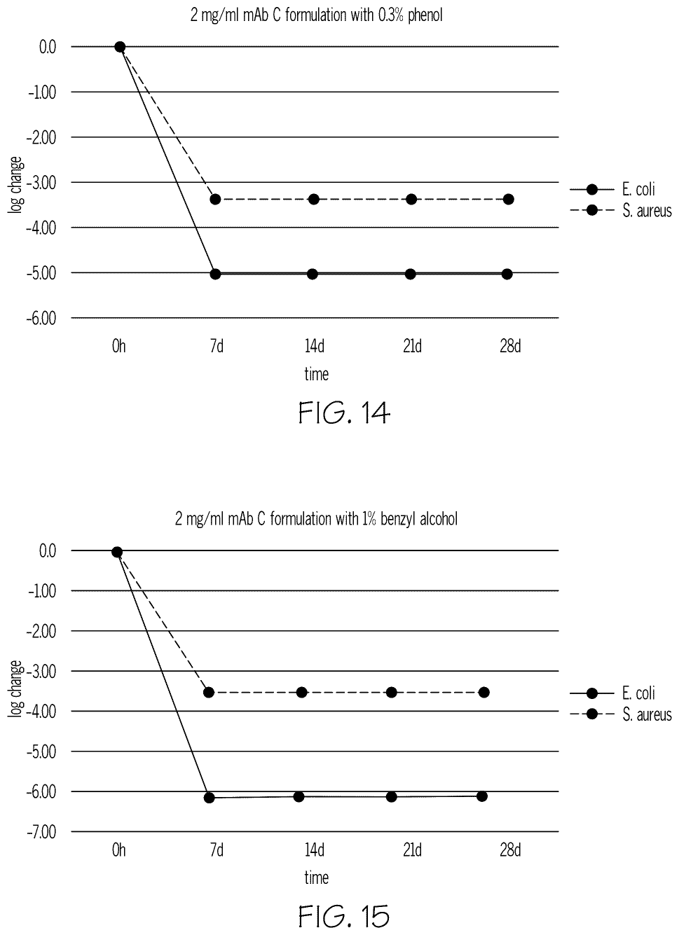

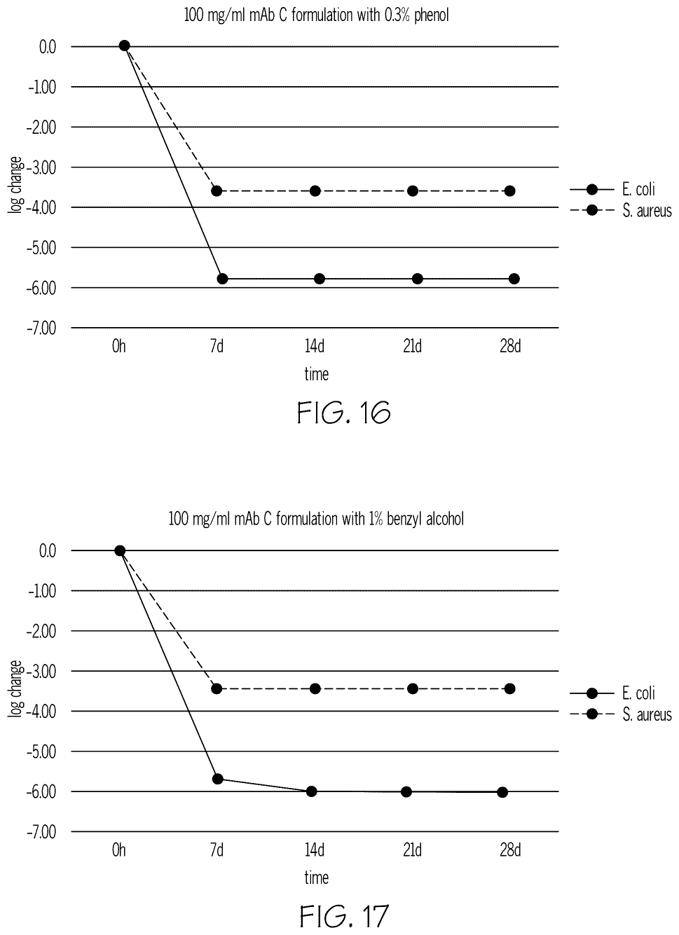

TABLE 3
Study Test Conditions and Sample Timepoints at 20-25° C.

| Drug Product | | | | | | | |
|---|---|---|---|---|---|---|---|
| (DP) | DP Concentration | Testing | | | | | |
| Formulation | (mg/mL) | Organism | Sample Timepoints | | | | |
| Formulation | 120 | Escherichia | 0 hours | 7 days | 14 days | 21 days | 28 days |
| 1 | | coli | | | | | |
| (FIG. 10) | | (ATCC8739) | | | | | |
| | | Staphylococcus | 0 hours | 7 days | 14 days | 21 days | 28 days |
| | | aureus (ATCC | | | | | |
| | | 6538) | | | | | |
| Formulation | 120 | Escherichia | 0 hours | 7 days | 14 days | 21 days | 28 days |
| 2 | | coli | | | | | |
| (FIG. 11) | | (ATCC8739) | | | | | |
| | | Staphylococcus | 0 hours | 7 days | 14 days | 21 days | 28 days |
| | | aureus (ATCC | | | | | |
| | | 6538) | | | | | |
| Formulation | 200 | Escherichia | 0 hours | 7 days | 14 days | 21 days | 28 days |
| 3 | | coli | | | | | |
| (FIG. 12) | | (ATCC8739) | | | | | |
| | | Staphylococcus | 0 hours | 7 days | 14 days | 21 days | 28 days |
| | | aureus (ATCC | | | | | |
| | | 6538) | | | | | |
| Formulation | 200 | Escherichia | 0 hours | 7 days | 14 days | 21 days | 28 days |
| 4 | | coli | | | | | |
| (FIG. 13) | | (ATCC8739) | | | | | |
| | | Staphylococcus | 0 hours | 7 days | 14 days | 21 days | 28 days |
| | | aureus (ATCC | | | | | |
| | | 6538) | | | | | |
| Formulation | 2 | Escherichia | 0 hours | 7 days | 14 days | 21 days | 28 days |
| 5 | | coli | | | | | |
| (FIG. 14) | | (ATCC8739) | | | | | |
| | | Staphylococcus | 0 hours | 7 days | 14 days | 21 days | 28 days |
| | | aureus (ATCC | | | | | |
| | | 6538) | | | | | |
| Formulation | 2 | Escherichia | 0 hours | 7 days | 14 days | 21 days | 28 days |
| 6 | | coli | | | | | |
| (FIG. 15) | | (ATCC8739) | | | | | |
| | | Staphylococcus | 0 hours | 7 days | 14 days | 21 days | 28 days |
| | | aureus (ATCC | | | | | |
| | | 6538) | | | | | |
| Formulation | 100 | Escherichia | 0 hours | 7 days | 14 days | 21 days | 28 days |
| 7 | | coli | | | | | |
| (FIG. 16) | | (ATCC8739) | | | | | |
| | | Staphylococcus | 0 hours | 7 days | 14 days | 21 days | 28 days |
| | | aureus (ATCC | | | | | |
| | | 6538) | | | | | |
| Formulation | 100 | Escherichia | 0 hours | 7 days | 14 days | 21 days | 28 days |
| 8 | | coli | | | | | |
| (FIG. 17) | | (ATCC8739) | | | | | |
| | | Staphylococcus | 0 hours | 7 days | 14 days | 21 days | 28 days |
| | | aureus (ATCC | | | | | |
| | | 6538) | | | | | |

### Data Analysis

Following 5 days of incubation, plates were removed from the incubator and the colonies were counted using an automated plate reader, or by hand if necessary. The number of colonies between the two plates were averaged for the final number of colony forming units.

The log change was calculated based on Equation 1, where Tₓ is the sample microbial recovery at the specified time point and T₀ is the microbial recovery of the sample at 0 hours:

$$Log change = Log ( sample {T}_{x} ) - Log ( {T}_{0} ) \qquad Equation 1$$

### Results and Discussion

Formulations 1 through 8 tested for the method qualification had a target recovery between 50% and 200% for both organisms tested (E. coli and S. aureus), indicating that there is no interference of the formulations in the growth and recovery of the two organisms. Table 4 summarizes the method qualification results of Formulations 1 through 8.

TABLE 4
Method Qualification Results of Formulations 1 through 8.

| | | Sample | Control | Target % | |
|---|---|---|---|---|---|
| Drug | | Average | Average | Recovery | Pass/ |
| Product | Microorganism | CFU | CFU | (50-200%) | Fail |
| Formulation | Escherichia coli | 92 | 110 | 83.63 | Pass |
| 1 | Staphylococcus | 66 | 57 | 115.79 | Pass |
| | aureus | | | | |
| Formulation | Escherichia coli | 78 | 110 | 70.45 | Pass |
| 2 | Staphylococcus | 67 | 57 | 116.67 | Pass |
| | aureus | | | | |
| Formulation | Escherichia coli | 65 | 101 | 64.68 | Pass |
| 3 | Staphylococcus | 72 | 61 | 117.21 | Pass |
| | aureus | | | | |
| Formulation | Escherichia coli | 82 | 101 | 81.09 | Pass |
| 4 | Staphylococcus | 62 | 61 | 100.82 | Pass |
| | aureus | | | | |
| Formulation | Escherichia coli | 93 | 73 | 128.28 | Pass |
| 5 | Staphylococcus | 49 | 60 | 82.35 | Pass |
| | aureus | | | | |
| Formulation | Escherichia coli | 121 | 112 | 108.04 | Pass |
| 6 | Staphylococcus | 49 | 64 | 76.56 | Pass |
| | aureus | | | | |
| Formulation | Escherichia coli | 72 | 68 | 106.67 | Pass |
| 7 | Staphylococcus | 65 | 46 | 141.30 | Pass |
| | aureus | | | | |
| Formulation | Escherichia coli | 124 | 68 | 183.70 | Pass |
| 8 | Staphylococcus | 53 | 46 | 115.22 | Pass |
| | aureus | | | | |

FIG. 10 depicts a graphical illustration of the bioburden count (CFU/mL) of Formulation 1. The graph shows the log change in bacteria (E. coli and S. aureus) in the formulation at 20-25° C. over a 28 day period. Table 5 summarizes the collected data points depicted in FIG. 10.

TABLE 5
Formulation 1 incubated at 20-25° C. over a 28 day period

| | DP Formulation 1 | | | | | |
|---|---|---|---|---|---|---|
| | 120 mg/mL mAb A + mAb B, 10 mM histidine, 8% (w/v) | | | | | |
| | sucrose, 0.1% (w/v) polysorbate 80, 3 mg/ml phenol | | | | | |
| | | | | Log₁₀ | | |
| Challenge | | Temperature | Titer | Microbial | Log₁₀ | Meets USP |
| Microbe | Time | (° C.) | (CFU/mL) | Count | Growth | 51 Criteria¹ |
| Escherichia | 0 d | 20-25° C. | 8020000 | 6.90 | 0 | Yes |
| coli | 7 d | 20-25° C. | 41 | 1.61 | −5.29 | Yes |
| | 14 d | 20-25° C. | 2 | 0.30 | −6.60 | Yes |
| | 21 d | 20-25° C. | 2 | 0.30 | −6.60 | Yes |
| | 28 d | 20-25° C. | 2 | 0.30 | −6.60 | Yes |
| Staphylococcus | 0 d | 20-25° C. | 5590 | 3.75 | 0 | Yes |
| aureus | 7 d | 20-25° C. | 2 | 0.30 | −3.45 | Yes |
| | 14 d | 20-25° C. | 2 | 0.30 | −3.45 | Yes |
| | 21 d | 20-25° C. | 2 | 0.30 | −3.45 | Yes |
| | 28 d | 20-25° C. | 2 | 0.30 | −3.45 | Yes |

¹Yes = greater than 1 log reduction at day 7 and greater than 3 log reduction at day 14-28

FIG. 11 depicts a graphical illustration of the bioburden count (CFU/mL) of Formulation 2. The graph shows the log change in bacteria (E. coli and S. aureus) in the formulation at 20-25° C. over a 28 day period. Table 6 summarizes the collected data points depicted in FIG. 11.

TABLE 6
Formulation 2 incubated at 20-25° C. over a 28 day period

| | DP Formulation 2 | | | | | |
|---|---|---|---|---|---|---|
| | 200 mg/mL mAb D, 20 mM histidine, pH 5.8, 100 mM arginine hydrochlori de, 2% | | | | | |
| | (w/v) sucrose, 0.15% (w/v) polysorbate 80, 3 mg/ml phenol | | | | | |
| | | | | Log₁₀ | | |
| Challenge | | Temperature | Titer | Microbial | Log₁₀ | Meets USP |
| Microbe | Time | (° C.) | (CFU/mL) | Count | Growth | 51 Criteria¹ |
| Escherichia coli | 0 d | 20-25° C. | 6860000 | 6.84 | 0 | Yes |
| | 7 d | 20-25° C. | 2 | 0.30 | −6.54 | Yes |
| | 14 d | 20-25° C. | 3 | 0.30 | −6.36 | Yes |
| | 21 d | 20-25° C. | 2 | 0.30 | −6.54 | Yes |
| | 28 d | 20-525° C. | 2 | 0.30 | −6.54 | Yes |
| Staphylococcus | 0 d | 20-25° C. | 5140 | 3.71 | 0 | Yes |
| aureus | 7 d | 20-25° C. | 2 | 0.30 | −3.41 | Yes |
| | 14 d | 20-25° C. | 2 | 0.30 | −3.41 | Yes |
| | 21 d | 20-25° C. | 2 | 0.30 | −3.41 | Yes |
| | 28 d | 20-25° C. | 2 | 0.30 | −3.41 | Yes |

¹Yes = greater than 1 log reduction at day 7 and greater than 3 log reduction at day 14-28

FIG. 12 depicts a graphical illustration of the bioburden count (CFU/mL) of Formulation 3. The graph shows the log change in bacteria (E. coli and S. aureus) in the formulation at 20-25° C. over a 28 day period. Table 7 summarizes the collected data points depicted in FIG. 12.

TABLE 7
Formulation 3 incubated at 20-25° C. over a 28 day period

| | DP Formulation 3 | | | | | |
|---|---|---|---|---|---|---|
| | 200 mg/mL mAb D, 20 mM histidine, pH 5.8, 100 mM arginine hydrochloride, 2% | | | | | |
| | (w/v) sucrose, 0.15% (w/v) polysorbate 80, 3 mg/ml phenol | | | | | |
| | | | | Log₁₀ | | |
| Challenge | | Temperature | Titer | Microbial | Log₁₀ | Meets USP |
| Microbe | Time | (° C.) | (CFU/mL) | Count | Growth | 51 Criteria¹ |
| Escherichia | 0 d | 20-25° C. | 6650000 | 6.82 | 0 | Yes |
| coli | 7 d | 20-25° C. | 2 | 0.30 | −6.52 | Yes |
| | 14 d | 20-25° C. | 2 | 0.30 | −6.52 | Yes |
| | 21 d | 20-25° C. | 2 | 0.30 | −6.52 | Yes |
| | 28 d | 20-25° C. | 2 | 0.30 | −6.52 | Yes |
| Staphylococcus | 0 d | 20-25° C. | 7220 | 3.86 | 0 | Yes |
| aureus | 7 d | 20-25° C. | 2 | 0.30 | −3.56 | Yes |
| | 14 d | 20-25° C. | 2 | 0.30 | −3.56 | Yes |
| | 21 d | 20-25° C. | 2 | 0.30 | −3.56 | Yes |
| | 28 d | 20-25° C. | 2 | 0.30 | −3.56 | Yes |

¹Yes = greater than 1 log reduction at day 7 and greater than 3 log reduction at day 14-28

FIG. 13 depicts a graphical illustration of the bioburden count (CFU/mL) of Formulation 4. The graph shows the log change in bacteria (E. coli and S. aureus) in the formulation at 20-25-C over a 28 day period. Table 8 summarizes the collected data points depicted in FIG. 13.

TABLE 8
Formulation 4 incubated at 20-25° C. over a 28 day period

| | DP Formulation 4 | | | | | |
|---|---|---|---|---|---|---|
| | 200 mg/mL mAb D, 20 mM histidine, pH 5.8, 100 mM arginine hydrochloride, 2% | | | | | |
| | (w/v) sucrose, 0.15% (w/v) polysorbate 80, 10 mg/ml benzyl alcohol | | | | | |
| | | | | Log₁₀ | | |
| Challenge | | Temperature | Titer | Microbial | Log₁₀ | Meets USP |
| Microbe | Time | (° C.) | (CFU/mL) | Count | Growth | 51 Criteria¹ |
| Escherichia | 0 d | 20-25° C. | 230000 | 5.36 | 0 | Yes |
| coli | 7 d | 20-25° C. | 2 | 0.30 | −5.08 | Yes |
| | 14 d | 20-25° C. | 2 | 0.30 | −5.08 | Yes |
| | 21 d | 20-25° C. | 2 | 0.30 | −5.08 | Yes |
| | 28 d | 20-25° C. | 2 | 0.30 | −5.08 | No |
| Staphylococcus | 0 d | 20-25° C. | 5020 | 3.70 | 0 | Yes |
| aureus | 7 d | 20-25° C. | 2 | 0.30 | −3.40 | Yes |
| | 14 d | 20-25° C. | 2 | 0.30 | −3.40 | Yes |
| | 21 d | 20-25° C. | 2 | 0.30 | −3.40 | Yes |
| | 28 d | 20-25° C. | 2 | 0.30 | −3.40 | Yes |

¹Yes = greater than 1 log reduction at day 7 and greater than 3 log reduction at day 14-28

FIG. 14 depicts a graphical illustration of the bioburden count (CFU/mL) of Formulation 5. The graph shows the log change in bacteria (E. coli and S. aureus) in the formulation at 20-25° C. over a 28 day period. Table 9 summarizes the collected data points depicted in FIG. 14.

TABLE 9
Formulation 5 incubated at 20-25° C. over a 28 day period

| | DP Formulation 5 | | | | | |
|---|---|---|---|---|---|---|
| | 2 mg/mL mAb C, 10 mM Histidine, 0.1% (w/v) polysorbate 80, | | | | | |
| | 10% (w/v) sucrose, 3 mg/ml phenol | | | | | |
| | | | | Log₁₀ | | |
| Challenge | | Temperature | Titer | Microbial | Log₁₀ | Meets USP |
| Microbe | Time | (° C.) | (CFU/mL) | Count | Growth | 51 Criteria¹ |
| Escherichia | 0 d | 20-25° C. | 2820000 | 6.45 | 0 | Yes |
| coli | 7 d | 20-25° C. | 21 | 1.32 | −5.13 | Yes |
| | 14 d | 20-25° C. | 2 | 0.30 | −6.15 | Yes |
| | 21 d | 20-25° C. | 2 | 0.30 | −6.15 | Yes |
| | 28 d | 20-25° C. | 2 | 0.30 | −6.15 | Yes |
| Staphylococcus | 0 d | 20-25° C. | 5970 | 3.78 | 0 | Yes |
| aureus | 7 d | 20-25° C. | 2 | 0.30 | −3.47 | Yes |
| | 14 d | 20-25° C. | 2 | 0.30 | −3.47 | Yes |
| | 21 d | 20-25° C. | 2 | 0.30 | −3.47 | Yes |
| | 28 d | 20-25° C. | 2 | 0.30 | −3.47 | Yes |

¹Yes = greater than 1 log reduction at day 7 and greater than 3 log reduction at day 14-28

FIG. 15 depicts a graphical illustration of the bioburden count (CFU/mL) of Formulation 6. The graph shows the log change in bacteria (E. coli and S. aureus) in the formulation at 20-25° C. over a 28 day period. Table 10 summarizes the collected data points depicted in FIG. 15.

TABLE 10
Formulation 6 incubated at 20-25° C. over a 28 day period

| | DPFormulation 6 | | | | | |
|---|---|---|---|---|---|---|
| | 2 mg/mL mAb C, 10 mM Histidine, 0.1% (w/v) polysorbate 80, | | | | | |
| | 10% (w/v) sucrose, 10 mg/ml benzyl alcohol | | | | | |
| | | | | Log₁₀ | | |
| Challenge | | Temperature | Titer | Microbial | Log₁₀ | Meets USP |
| Microbe | Time | (° C.) | (CFU/mL) | Count | Growth | 51 Criteria¹ |
| Escherichia | 0 d | 20-25° C. | 2800000 | 6.45 | 0 | Yes |
| coli | 7 d | 20-25° C. | 2 | 0.30 | −6.15 | Yes |
| | 14 d | 20-25° C. | 2 | 0.30 | −6.15 | Yes |
| | 21 d | 20-25° C. | 2 | 0.30 | −6.15 | Yes |
| | 28 d | 20-25° C. | 2 | 0.30 | 6.15 | Yes |
| Staphylococcus | 0 d | 20-25° C. | 6150 | 3.79 | 0 | Yes |
| aureus | 7 d | 20-25° C. | 2 | 0.30 | −3.49 | Yes |
| | 14 d | 20-25° C. | 2 | 0.30 | −3.49 | Yes |
| | 21 d | 20-25° C. | 2 | 0.30 | −3.49 | Yes |
| | 28 d | 20-25° C. | 2 | 0.30 | −3.49 | Yes |

¹Yes = greater than 1 log reduction at day 7 and greater than 3 log reduction at day 14-28

FIG. 16 depicts a graphical illustration of the bioburden count (CFU/mL) of Formulation 7. The graph shows the log change in bacteria (E. coli and S. aureus) in the formulation at 20-25° C. over a 28 day period. Table 11 summarizes the collected data points depicted in FIG. 16.

TABLE 11
Formulation 7 incubated at 20-25° C. over a 28 day period

| | DP Formulation 7 | | | | | |
|---|---|---|---|---|---|---|
| | 100 mg/mL mAb C, 10 mM Histidine, 0.1% (w/v) polysorbate 80, | | | | | |
| | 10% (w/v) sucrose, 3 mg/ml phenol | | | | | |
| | | | | Log₁₀ | | |
| Challenge | | Temperature | Titer | Microbial | Log₁₀ | Meets USP |
| Microbe | Time | (° C.) | (CFU/mL) | Count | Growth | 51 Criteria¹ |
| Escherichia | 0 d | 20-25° C. | 1200000 | 6.08 | 0 | Yes |
| coli | 7 d | 20-25° C. | 2 | 0.30 | −5.78 | Yes |
| | 14 d | 20-25° C. | 2 | 0.30 | −5.78 | Yes |
| | 21 d | 20-25° C. | 2 | 0.30 | −5.78 | Yes |
| | 28 d | 20-25° C. | 2 | 0.30 | −5.78 | Yes |
| Staphylococcus | 0 d | 20-25° C. | 6720 | 3.83 | 0 | Yes |
| aureus | 7 d | 20-25° C. | 2 | 0.30 | −3.53 | Yes |
| | 14 d | 20-25° C. | 2 | 0.30 | −3.53 | Yes |
| | 21 d | 20-25° C. | 2 | 0.30 | −3.53 | Yes |
| | 28 d | 20-25° C. | 2 | 0.30 | −3.53 | Yes |

¹Yes = greater than 1 log reduction at day 7 and greater than 3 log reduction at day 14-28

FIG. 17 depicts a graphical illustration of the bioburden count (CFU/mL) of Formulation 8. The graph shows the log change in bacteria (E. coli and S. aureus) in the formulation at 20-25° C. over a 28 day period. Table 12 summarizes the collected data points depicted in FIG. 17.

TABLE 12
Formulation 8 incubated at 20-25° C. over a 28 day period

| | DP Formulation 8 | | | | | |
|---|---|---|---|---|---|---|
| | 100 mg/mL mAb C, 10 mM Histidine, 0.1% (w/v) polysorbate 80, 10% (w/v) | | | | | |
| | sucrose, 3 mg/ml phenol | | | | | |
| | | | | Log₁₀ | | |
| Challenge | | | Titer | Microbial | Log₁₀ | Meets USP |
| Microbe | Time | Temperature | (CFU/mL) | Count | Growth | 51 Criteria¹ |
| Escherichia | 0 d | 20-25° C. | 2470000 | 6.39 | 0 | Yes |
| coli | 7 d | 20-25° C. | 4 | 0.60 | −5.79 | Yes |
| | 14 d | 20-25° C. | 2 | 0.30 | −6.09 | Yes |
| | 21 d | 20-25° C. | 2 | 0.30 | −6.09 | Yes |
| | 28 d | 20-25° C. | 2 | 0.30 | −6.09 | Yes |
| | 0 d | 20-25° C. | 6050 | 3.78 | 0 | Yes |
| Staphylococcus | 7 d | 20-25° C. | 2 | 0.30 | −3.48 | Yes |
| aureus | 14 d | 20-25° C. | 2 | 0.30 | −3.48 | Yes |
| | 21 d | 20-25° C. | 2 | 0.30 | −3.48 | Yes |
| | 28 d | 20-25° C. | 2 | 0.30 | −3.48 | Yes |

¹Yes = greater than 1 log reduction at day 7 and greater than 3 log reduction at day 14-28

### Deviation

The initial experiments for Escherichia coli did not have a CFU value high enough to reach a readable 3 log reduction. This was partly believed to be caused by difficulty in reading the initial plates due to the amount of growth. The experiment was repeated to reach a higher initial CFU. Additionally, the 1 mL sample was split into 10, 0.1 mL aliquots, which were plated into 10 separate petri dishes for plate counting purposes. Tempered agar was then poured over these samples and the sum of the 10 plates were used to calculate the initial CFU.

### Positive Control Groups:

Four formulations (i.e., Positive Control Formulations 1-4) containing no preservatives were used as a control and showed continued growth for at least one timepoint beyond when full sterilization occurred in the same formulations containing either preservative. Tables 13-17 summarize the collected data points for Positive Control Formulations 1-4 containing no preservatives.

Positive Control Formulation 1 is summarized in Table 13.

Table 13 summarizes the collected data of Positive Control Formulation 1 stored with no preservative at 20-25° C. over a 28 day period.

TABLE 13
Positive Control Formulation 1 stored at 20-25° C. over a 28 day period

| | 120 mg/mL mAb A + mAb B, | | | | |
|---|---|---|---|---|---|
| | 10mM histidine, 8% (w/v) sucrose, | | | | |
| DP | 0.1% (w/v) polysorbate 80 | | | | |
| Formulation 1 | | | | Log₁₀ | |
| Challenge | | Temperature | Titer | Microbial | Log₁₀ |
| Microbe | Time | (° C.) | (CFU/mL) | Count | Growth |
| Escherichia | 0 d | 20 - 25° C. | 655000 | 6.94 | 0 |
| coli | 7d | 20 - 25° C. | 25000000 | 7.81 | 0.88 |
| | 14d | 20 - 25° C. | 300000000 | 8.00 | 1.06 |
| | 21d | 20 - 25° C. | 8000000 | 6.30 | −0.64 |
| | 28d | 20 - 25° C. | 9000000 | 6.70 | −0.24 |
| Staphylococcus | 0 d | 20 - 25° C. | 746000 | 5.87 | 0 |
| aureus | 7d | 20 - 25° C. | 7000000 | 6.85 | 0.97 |
| | 14d | 20 - 25° C. | 50000000 | 7.70 | 1.83 |
| | 21d | 20 - 25° C. | 3000000 | 6.48 | 0.60 |
| | 28d | 20 - 25° C. | 18000000 | 7.26 | 1.38 |

Table 14 summarizes the collected data of Positive Control Formulation 2 stored with no preservative at 20-25° C. over a 28 day period.

TABLE 14
Positive Control Formulation 2 stored at 20-25° C. over a 28 day period

| | 200mg/mL mAb D, 20mM histidine, | | | | |
|---|---|---|---|---|---|
| | pH 5.8, 100mM arginine | | | | |
| | hydrochloride, 2% (w/v) sucrose, | | | | |
| DP | 0.15% (w/v) polysorbate 80 | | | | |
| Formulation 2 | | | | Log₁₀ | |
| Challenge | | Temperature | Titer | Microbial | Log₁₀ |
| Microbe | Time | (° C.) | (CFU/mL) | Count | Growth |
| Escherichia | 0 d | 20 - 25° C. | 8580000 | 6.93 | 0 |
| coli | 7d | 20 - 25° C. | 154000000 | 8.19 | 1.25 |
| | 14d | 20 - 25° C. | 102000000 | 8.01 | 1.08 |
| | 21d | 20 - 25° C. | 14000000 | 7.15 | 0.21 |
| | 28d | 20 - 25° C. | 138000000 | 8.14 | 1.21 |
| Staphylococcus | 0 d | 20 - 25° C. | 706000 | 5.85 | 0 |
| aureus | 7d | 20 - 25° C. | 2000000 | 6.30 | 0.45 |
| | 14d | 20 - 25° C. | 184000000 | 8.26 | 2.42 |
| | 21d | 20 - 25° C. | 88000000 | 7.94 | 2.10 |
| | 28d | 20 - 25° C. | 136000000 | 8.13 | 2.28 |

Table 15 summarizes the collected data of Positive Control Formulation 3 stored with no preservative at 20-25° C. over a 28 day period.

TABLE 15
Positive Control Formulation 3 stored at 20-25° C. over a 28 day period

| | 2mg/mL mAb C, 10 mM histidine, | | | | |
|---|---|---|---|---|---|
| | 0.1% (w/v) polysorbate 80, | | | | |
| DP | 10% (w/v) sucrose | | | | |
| Formulation 3 | | | | Log₁₀ | |
| Challenge | | Temperature | Titer | Microbial | Log₁₀ |
| Microbe | Time | (° C.) | (CFU/mL) | Count | Growth |
| Escherichia | 0 d | 20 - 25° C. | 655000 | 5.82 | 0 |
| coli | 7d | 20 - 25° C. | 25000000 | 7.40 | 1.58 |
| | 14d | 20 - 25° C. | 30000000 | 7.48 | 1.66 |
| | 21d | 20 - 25° C. | 8000000 | 6.90 | 1.09 |
| | 28d | 20 - 25° C. | 9000000 | 6.95 | 1.14 |
| Staphylococcus | 0 d | 20 - 25° C. | 550000 | 5.74 | 0 |
| aureus | 7d | 20 - 25° C. | 21000000 | 7.32 | 1.58 |
| | 14d | 20 - 25° C. | 33000000 | 7.52 | 1.78 |
| | 21d | 20 - 25° C. | 6000000 | 6.78 | 1.04 |
| | 28d | 20 - 25° C. | 34000000 | 7.53 | 1.79 |

Table 16 summarizes the collected data of Positive Control Formulation 4 stored with no preservative at 20-25° C. over a 28 day period.

TABLE 16
Positive Control Formulation 4 stored at 20-25° C. over a 28 day period

| | 100mg/mL mAb C, 10mM histidine, | | | | |
|---|---|---|---|---|---|
| | 0.1% (w/v) polysorbate 80, | | | | |
| DP | 10% (w/v) sucrose | | | | |
| Formulation | | | | Log₁₀ | |
| Challenge | | Temperature | Titer | Microbial | Log₁₀ |
| Microbe | Time | (° C.) | (CFU/mL) | Count | Growth |
| Escherichia | 0 d | 20 - 25° C. | 552000 | 5.74 | 0 |
| coli | 7d | 20 - 25° C. | 26000000 | 7.41 | 1.67 |
| | 14d | 20 - 25° C. | 76000000 | 7.88 | 2.14 |
| | 21d | 20 - 25° C. | 8000000 | 6.90 | 1.16 |
| | 28d | 20 - 25° C. | 17000000 | 7.23 | 1.49 |
| Staphylococcus | 0 d | 20 - 25° C. | 854000 | 5.93 | 0 |
| aureus | 7d | 20 - 25° C. | 25000000 | 7.40 | 1.47 |
| | 14d | 20 - 25° C. | 131000000 | 8.12 | 2.19 |
| | 21d | 20 - 25° C. | 82000000 | 7.91 | 1.98 |
| | 28d | 20 - 25° C. | 16000000 | 7.20 | 1.27 |

### CONCLUSION

For the gram-positive representative, Staphylococcus aureus, all drug product formulations, reached a greater than 1 log reduction by the₇th day of the study and a greater than 3 log reduction for the remainder of the study.

For the gram-negative representative, Escherichia coli, all drug product formulations, reached a greater than 1 log reduction by the₇th day of the study and a greater than 3 log reduction for the remainder of the study.

The data in FIGS. 10-17 demonstrate that for the gram-positive representative, Staphylococcus aureus, all drug product formulations, reached a greater than 1 log reduction by the seventh day of the study and a greater than 3 log reduction for the remainder of the study. For the gram-negative representative, Escherichia coli, all drug product formulations, reached a greater than 1 log reduction by the seventh day of the study and a greater than 3 log reduction for the remainder of the study.

### Additional Testing

Tables 17 through 40 summarize collected data points from additional testing of the effect of preservatives on drug product formulations, including formulation turbidity, HMW complex formation, charge variant formation, and subvisible particle formation under various stress conditions. Testing was conducted for eight target formulations (i.e., Target Formulations 1-8) containing preservatives (phenol or benzyl alcohol).

Target Formulation 1 is summarized in Tables 17-19.

Table 17 summarizes the collected data of Target Formulation 1 stored with 0.3% phenol at 5° C. for 24 months.

TABLE 17
Target Formulation 1 with 0.3% phenol stored at 5° C. for 24 months

| | | Target Formulation | | | | | | |
|---|---|---|---|---|---|---|---|---|
| | | 120 mg/mL mAb A + mAb B formulation with 0.3% phenol | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.00 | 0.01 | 0.01 | 0.01 |
| pH | | 6.3 | 6.3 | 6.2 | 6.2 | 6.2 | 6.3 | 6.2 |
| % mAb Recovered by RP-UPLC | | 100 | 97 | 98 | 105 | 101 | 97 | 95 |
| Purity by SE-UPLC | % HMW | 1.1 | 1.1 | 1.1 | 1.1 | 1.3 | 1.4 | 1.4 |
| | % Main | 97.9 | 97.9 | 97.7 | 97.8 | 97.4 | 97.2 | 97.3 |
| | % LMW | 1.0 | 1.0 | 1.2 | 1.0 | 1.3 | 1.3 | 1.3 |
| MFI (# particles/mL) | 2-10 μm | 1225 | 658 | — | 624 | 2796 | — | 113 |
| (aspect ratio ≤ 0.85 | ≥10 μm | 66 | 79 | — | 69 | 181 | — | 10 |
| applied) | ≥25 μm | 7 | 7 | — | 10 | 13 | — | 2 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 18 summarizes the collected data of Target Formulation 1 stored with 0.3% phenol at 25° C. for 6 months.

TABLE 18
Target Formulation 1 with 0.3% phenol stored at 25° C. for 6 months

| | | Target Formulation | | | | | |
|---|---|---|---|---|---|---|---|
| | | 120 mg/mL mAb A + mAb B formulation with 0.3% phenol | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.01 | 0.01 | 0.01 | 0.02 |
| pH | | 6.3 | 6.2 | 6.3 | 6.2 | 6.2 | 6.3 |
| % mAb recovered by RP-UPLC | | 100 | 97 | 103 | 96 | 98 | 104 |
| Purity by SE-UPLC | % HMW | 1.1 | 1.3 | 1.3 | 1.4 | 1.7 | 2.1 |
| | % Main | 97.9 | 97.7 | 97.6 | 97.2 | 96.7 | 95.5 |
| | % LMW | 1.0 | 1.1 | 1.1 | 1.4 | 1.6 | 2.4 |
| MFI (# particles/mL) | 2-10 μm | 1225 | — | 925 | — | — | 475 |
| (aspect ratio ≤ 0.85 | ≥10 μm | 66 | — | 215 | — | — | 87 |
| applied) | ≥25 μm | 7 | — | 10 | — | — | 15 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 19 summarizes the collected data of Target Formulation 1 stored with 0.3% phenol at 40° C. and 45° C. for 3 months.

TABLE 19
Target Formulation 1 with 0.3% phenol stored at 40° C. and 45° C. for 3 months

| | | Target Formulation | | | | | | | | |
|---|---|---|---|---|---|---|---|---|---|---|
| | | 120 mg/mL mAb A + mAb B formulation with 0.3% phenol | | | | | | | | |
| | | | Length of Storage at 40° C. | | | | Length of Storage at 45° C. | | | |
| | | | (months) | | | | (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Pass |
| Increase in Turbidity (OD 405 nm) | | 0.00 | 0.01 | 0.02 | 0.04 | 0.05 | 0.02 | 0.04 | 0.06 | 0.10 |
| pH | | 6.3 | 6.2 | 6.3 | 6.3 | 6.2 | 6.3 | 6.3 | 6.3 | 6.2 |
| % mAb Recovered by RP-UPLC | | 100 | 98 | 97 | 97 | 98 | 98 | 100 | 94 | 94 |
| Purity by SE-UPLC | % HMW | 1.1 | 1.7 | 2.2 | 2.9 | 5.4 | 2.4 | 3.3 | 4.9 | 8.6 |
| | % Main | 97.9 | 96.8 | 95.9 | 93.3 | 90.9 | 95.7 | 93.8 | 89.1 | 84.2 |
| | % LMW | 1.0 | 1.5 | 2.0 | 3.8 | 3.7 | 1.9 | 2.9 | 6.0 | 7.3 |
| MFI (# particles/mL) | 2-10 μm | 1225 | — | 287 | — | 563 | — | 638 | — | 796 |
| (aspect ratio ≤ 0.85 | ≥10 μm | 66 | — | 54 | — | 64 | — | 109 | — | 66 |
| applied) | ≥25 μm | 7 | — | 10 | — | 7 | — | 8 | — | 5 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Target Formulation 2 is summarized in Tables 20-22.

Table 20 summarizes the collected data of Target Formulation 2 stored with 1% benzyl alcohol at 5° C. for 24 months.

TABLE 20
Target Formulation 2 with 1% benzyl alcohol stored at 5° C. for 24 months

| | | Target Formulation | | | | | | |
|---|---|---|---|---|---|---|---|---|
| | | 120 mg/mL mAb A + mAb B formulation with 1% benzyl alcohol | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.00 | 0.01 | 0.01 | 0.01 |
| pH | | 6.3 | 6.3 | 6.2 | 6.3 | 6.3 | 6.3 | 6.3 |
| % mAb Recovered by RP-UPLC | | 100 | 105 | 103 | 108 | 108 | 101 | 99 |
| Purity by SE-UPLC | % HMW | 1.1 | 1.2 | 1.2 | 1.3 | 1.3 | 1.5 | 1.5 |
| | % Main | 97.9 | 97.8 | 97.4 | 97.6 | 97.9 | 97.2 | 97.2 |
| | % LMW | 1.0 | 1.0 | 1.4 | 1.1 | 0.9 | 1.2 | 1.2 |
| MFI (# particles/ mL) | 2-10 μm | 866 | 591 | — | 7362 | 3351 | — | 298 |
| (aspect ratio ≤ 0.85 | ≥10 μm | 82 | 38 | — | 2632 | 198 | — | 19 |
| applied) | ≥25 μm | 5 | 0 | — | 111 | 8 | — | 6 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 21 summarizes the collected data of Target Formulation 2 stored with 1% benzyl alcohol at 25° C. for 6 months.

TABLE 21
Target Formulation 2 with 1% benzyl alcohol stored at 25° C. for 6 months

| | | Target Formulation | | | | | |
|---|---|---|---|---|---|---|---|
| | | 120 mg/mL mAb A + mAb B formulation with 1% benzyl alcohol | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.01 | 0.01 | 0.01 | 0.01 | 0.02 |
| pH | | 6.3 | 6.3 | 6.3 | 6.2 | 6.2 | 6.3 |
| % mAb Recovered by RP-UPLC | | 100 | 105 | 102 | 104 | 105 | 106 |
| Purity by SE-UPLC | % HMW | 1.1 | 1.2 | 1.4 | 1.5 | 5.3 | 1.9 |
| | % Main | 97.9 | 97.8 | 97.5 | 97.3 | 93.0 | 85.1 |
| | % LMW | 1.0 | 1.0 | 1.1 | 1.2 | 1.7 | 13.1 |
| MFI (# particles/mL) | 2-10 μm | 866 | — | 640 | — | — | 200 |
| (aspect ratio ≤ 0.85 | ≥10 μm | 82 | — | 131 | — | — | 57 |
| applied) | ≥25 μm | 5 | — | 13 | — | — | 15 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 22 summarizes the collected data of Target Formulation 2 stored with 1% benzyl alcohol at 40° C. and 45° C. for 3 months.

TABLE 22
Target Formulation 2 with 1% benzyl alcohol stored at 40° C. and 45° C. for 3 months

| | | Target Formulation | | | | | | | | |
|---|---|---|---|---|---|---|---|---|---|---|
| | | 120 mg/mL mAb A + mAb B formulation with 1% benzyl alcohol | | | | | | | | |
| | | | Length of Storage at 40° C. | | | | Length of Storage at 45° C. | | | |
| | | | (months) | | | | (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Pass |
| Increase in Turbidity (OD 405 nm) | | 0.00 | 0.02 | 0.02 | 0.04 | 0.05 | 0.03 | 0.05 | 0.09 | 0.16 |
| pH | | 6.3 | 6.3 | 6.3 | 6.3 | 6.2 | 6.3 | 6.3 | 6.3 | 6.2 |
| % mAb Recovered by RP-UPLC | | 100 | 102 | 105 | 101 | 98 | 107 | 101 | 100 | 98 |
| Purity by SE-UPLC | % HMW | 1.1 | 2.0 | 2.6 | 3.4 | 4.7 | 3.4 | 5.2 | 8.3 | 7.2 |
| | % Main | 97.9 | 96.5 | 95.4 | 92.6 | 90.5 | 94.7 | 92.0 | 85.4 | 79.1 |
| | % LMW | 1.0 | 1.5 | 2.0 | 4.1 | 4.7 | 1.9 | 2.8 | 6.3 | 13.7 |
| MFI (# particles/ mL) | 2-10 μm | 866 | — | 292 | — | 1027 | — | 1020 | — | 706 |
| (aspect ratio ≤ 0.85 | ≥10 μm | 82 | — | 62 | — | 111 | — | 93 | — | 57 |
| applied) | ≥25 μm | 5 | — | 8 | — | 5 | — | 11 | — | 7 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Target Formulation 3 is summarized in Tables 23-25.

Table 23 summarizes the collected data of Target Formulation 3 stored with 0.3% phenol at 5° C. for 24 months.

TABLE 23
Target Formulation 3 with 0.3% phenol stored at 5° C. for 24 months

| | | Target Formulation | | | | | | |
|---|---|---|---|---|---|---|---|---|
| | | 200 mg/mL mAb D formulation with 0.3% phenol | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.00 | 0.00 | 0.01 | 0.01 |
| pH | | 5.9 | 5.9 | 5.8 | 5.8 | 5.9 | 5.9 | 5.8 |
| % mAb Recovered by RP-UPLC | | 100 | 101 | 92 | 104 | 100 | 101 | 107 |
| Purity by SE-UPLC | % HMW | 0.8 | 0.8 | 0.8 | 1.0 | 0.8 | 0.9 | 1.0 |
| | % Main | 98.9 | 98.9 | 98.8 | 98.1 | 98.7 | 98.7 | 98.5 |
| | % LMW | 0.3 | 0.3 | 0.4 | 0.9 | 0.4 | 0.4 | 0.5 |
| Charge Variant | % Acidic | 21.8 | 21.8 | 22.1 | NA* | 22.0 | 22.7 | 22.7 |
| Analysis by | % Main | 61.4 | 61.5 | 62.3 | NA* | 62.9 | 63.1 | 63.4 |
| CEX-UPLC | % Basic | 16.8 | 16.7 | 15.7 | NA* | 15.1 | 14.2 | 13.9 |
| MFI (# particles/mL) | 2-10 μm | 1824 | 2255 | — | 269 | 990 | — | 196 |
| (aspect ratio ≤ 0.85 | ≥10 μm | 100 | 292 | — | 71 | 85 | — | 27 |
| applied) | ≥25 μm | 10 | 58 | — | 0 | 6 | — | 10 |

CEX, cation exchange; HMW, high molecular weight; LMW, low molecular weight; MFI, Micro-Flow Imaging; NA, Not Available (*Data invalid due to error with sample injection); OD, optical density; RH, relative humidity; RP, Reverse Phase; SE, size exclusion; UPLC, ultra-performance liquid chromatography.

Table 24 summarizes the collected data of Target Formulation 3 stored with 0.3% phenol at 25° C. for 6 months.

TABLE 24
Target Formulation 3 with 0.3% phenol stored at 25° C. for 6 months

| | | Target Formulation | | | | | |
|---|---|---|---|---|---|---|---|
| | | 200 mg/mL mAb D formulation with 0.3% phenol | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.01 | 0.00 | 0.01 | 0.02 |
| pH | | 5.9 | 5.9 | 5.9 | 5.9 | 5.8 | 5.9 |
| % mAb Recovered by RP-UPLC | | 100 | 102 | 102 | 96 | 91 | 111 |
| Purity by SE-UPLC | % HMW | 0.8 | 0.9 | 0.9 | 1.0 | 1.1 | 1.2 |
| | % Main | 98.9 | 98.8 | 98.7 | 98.7 | 98.5 | 97.4 |
| | % LMW | 0.3 | 0.3 | 0.3 | 0.4 | 0.4 | 1.3 |
| Charge Variant Analysis | % Acidic | 21.8 | 21.4 | 21.9 | 22.9 | 24.2 | 28.0 |
| by CEX-UPLC | % Main | 61.4 | 61.9 | 62.0 | 62.8 | 62.6 | 14.2 |
| | % Basic | 16.8 | 16.8 | 16.1 | 14.2 | 13.3 | 57.8 |
| MFI (# particles/mL) | 2-10 μm | 1824 | — | 954 | — | — | 1261 |
| (aspect ratio ≤ 0.85 | ≥10 μm | 100 | — | 69 | — | — | 123 |
| applied) | ≥25 μm | 10 | — | 13 | — | — | 8 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 25 summarizes the collected data of Target Formulation 3 stored with 0.3% phenol at 40° C. and 45° C. for 3 months.

TABLE 25
Target Formulation 3 with 0.3% phenol stored at 40° C. and 45° C. for 3 months

| | | Target Formulation | | | | | | | | |
|---|---|---|---|---|---|---|---|---|---|---|
| | | 200 mg/mL mAb D formulation with 0.3% phenol | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Fail |
| Increase in Turbidity (OD 405 nm) | | 0.00 | 0.01 | 0.02 | 0.04 | 0.07 | 0.04 | 0.09 | 0.17 | 0.25 |
| pH | | 5.9 | 6.0 | 5.9 | 5.8 | 5.9 | 6.0 | 5.9 | 6.0 | 5.9 |
| % mAb Recovered by RP-UPLC | | 100 | 101 | 102 | 95 | 90 | 100 | 99 | 91 | 85 |
| Purity by SE-UPLC | % HMW | 0.8 | 2.2 | 3.7 | 6.9 | 10.9 | 10.7 | 18.3 | 26.1 | 29.5 |
| | % Main | 98.9 | 97.3 | 95.7 | 92.4 | 88.0 | 88.7 | 80.9 | 72.8 | 69.1 |
| | % LMW | 0.3 | 0.5 | 0.6 | 0.8 | 1.1 | 0.6 | 0.8 | 1.1 | 1.5 |
| Charge Variant Analysis | % Acidic | 21.8 | 23.8 | 28.4 | 40.8 | 51.8 | 27.4 | 39.6 | 692 | 69.9 |
| by CEX-UPLC | % Main | 61.4 | 60.9 | 56.9 | 46.4 | 36.2 | 58.2 | 46.8 | 19.8 | 9.2 |
| | % Basic | 16.8 | 15.3 | 14.7 | 12.8 | 12.0 | 14.4 | 13.6 | 11.1 | 20.9 |
| MFI (# particles/mL) | 2-10 μm | 1824 | — | 2357 | — | 1095 | — | 3711 | — | 21294 |
| (aspect ratio ≤ 0.85 | ≥10 μm | 100 | — | 211 | — | 152 | — | 342 | — | 3111 |
| applied) | ≥25 μm | 10 | — | 46 | — | 8 | — | 50 | — | 653 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Target Formulation 4 is summarized in Tables 26-28.

Table 26 summarizes the collected data of Target Formulation 4 stored with 1% benzyl alcohol at 5° C. for 24 months.

TABLE 26
Target Formulation 4 with 1% benzyl alcohol stored at 5° C. for 24 months

| | | Target Formulation | | | | | | |
|---|---|---|---|---|---|---|---|---|
| | | 200 mg/mL mAb D formulation with 1% benzyl alcohol | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.00 | 0.00 | 0.00 | 0.01 |
| pH | | 5.9 | 5.9 | 5.9 | 5.8 | 5.9 | 5.9 | 5.9 |
| % mAb Recovered by RP-UPLC | | 100 | 100 | 90 | 96 | 98 | 102 | 106 |
| Purity by SE-UPLC | % HMW | 0.8 | 0.8 | 0.8 | 0.9 | 0.9 | 1.0 | 0.9 |
| | % Main | 98.9 | 98.9 | 98.9 | 98.2 | 98.7 | 98.6 | 98.9 |
| | % LMW | 0.3 | 0.3 | 0.3 | 0.8 | 0.4 | 0.5 | 0.2 |
| Charge Variant Analysis | % Acidic | 20.3 | 20.5 | 21.4 | 21.8 | 22.1 | 21.1 | 20.8 |
| by CEX-UPLC | % Main | 63.8 | 63.5 | 63.3 | 62.3 | 66.3 | 65.5 | 66.3 |
| | % Basic | 15.9 | 16.0 | 15.4 | 15.9 | 13.6 | 13.4 | 12.9 |
| MFI (# particles/mL) | 2-10 μm | 1072 | 1887 | — | 192 | 694 | — | 949 |
| (aspect ratio ≤ 0.85 | ≥10 μm | 98 | 253 | — | 27 | 38 | — | 63 |
| applied) | ≥25 μm | 6 | 63 | — | 10 | 4 | — | 6 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 27 summarizes the collected data of Target Formulation 4 stored with 1% benzyl alcohol at 25-C for 6 months.

TABLE 27
Target Formulation 4 with 1% benzyl alcohol stored at 25° C. for 6 months

| | | Target Formulation | | | | | |
|---|---|---|---|---|---|---|---|
| | | 200 mg/mL mAb D formulation with 1% benzyl alcohol | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.00 | 0.00 | 0.01 | 0.02 |
| pH | | 5.9 | 5.9 | 5.9 | 5.9 | 5.9 | 5.9 |
| % mAb Recovered by RP-UPLC | | 100 | 100 | 101 | 95 | 91 | 110 |
| Purity by SE-UPLC | % HMW | 0.8 | 0.9 | 1.0 | 1.0 | 1.2 | 1.4 |
| | % Main | 98.9 | 98.7 | 98.7 | 98.6 | 98.5 | 97.7 |
| | % LMW | 0.3 | 0.3 | 0.3 | 0.4 | 0.4 | 0.9 |
| Charge Variant Analysis | % Acidic | 20.3 | 20.0 | 20.3 | 22.0 | 22.9 | 23.9 |
| by CEX-UPLC | % Main | 63.8 | 63.9 | 64.3 | 63.9 | 64.1 | 62.1 |
| | % Basic | 15.9 | 16.1 | 15.3 | 14.2 | 13.1 | 12.8 |
| MFI (# particles/mL) | 2-10 μm | 1072 | — | 954 | — | — | 1261 |
| (aspect ratio ≤ 0.85 | ≥10 μm | 98 | — | 69 | — | — | 123 |
| applied) | ≥25 μm | 6 | — | 13 | — | — | 8 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 28 summarizes the collected data of Target Formulation 4 stored with 1% benzyl alcohol at 40° C. and 45° C. for 3 months.

TABLE 28
Target Formulation 4 with 1% benzyl alcohol stored at 40° C. and 45° C. for 3 months

| | | Target Formulation | | | | | | | | |
|---|---|---|---|---|---|---|---|---|---|---|
| | | 200 mg/mL mAb D formulation with 1% benzyl alcohol | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Pass | Pass | Fail | Fail | Fail | Fail | Fai |
| Increase in Turbidity (OD 405 nm) | | 0.00 | 0.02 | 0.05 | 0.11 | 0.17 | 1.56 | NA | NA | NA |
| pH | | 5.9 | 5.9 | 5.9 | 5.9 | 5.9 | 6.0 | NA | NA | NA |
| % mAb Recovered by RP-UPLC | | 100 | 101 | 100 | 93 | 88 | NA | NA | NA | NA |
| Purity by SE-UPLC | % HMW | 0.8 | 5.6 | 9.7 | 15.5 | 19.0 | NA | NA | NA | NA |
| | % Main | 98.9 | 93.9 | 89.8 | 83.9 | 79.9 | NA | NA | NA | NA |
| | % LMW | 0.3 | 0.5 | 0.6 | 0.7 | 1.1 | NA | NA | NA | NA |
| Charge Variant Analysis | % Acidic | 20.3 | 21.5 | 26.3 | 38.6 | 49.9 | NA | NA | NA | NA |
| by CEX-UPLC | % Main | 63.8 | 63.9 | 60.7 | 49.1 | 39.9 | NA | NA | NA | NA |
| | % Basic | 15.9 | 14.5 | 13.1 | 12.4 | 10.7 | NA | NA | NA | NA |
| MFI (# particles/mL) | 2-10 μm | 1072 | — | 2357 | — | 1095 | NA | NA | NA | NA |
| (aspect ratio ≤ 0.85 | ≥10 μm | 98 | — | 211 | — | 152 | NA | NA | NA | NA |
| applied) | ≥25 μm | 6 | — | 46 | — | 8 | NA | NA | NA | NA |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Target Formulation 5 is summarized in Tables 29-31.

Table 29 summarizes the collected data of Target Formulation 5 stored with 0.3% phenol at 5° C. for 24 months.

TABLE 29
Target Formulation 5 with 0.3% phenol stored at 5° C. for 24 months

| | | Target Formulation | | | | | | |
|---|---|---|---|---|---|---|---|---|
| | | 2 mg/mL mAb C formulation with 0.3% phenol | | | | | | |
| | | | Length of Storage at 5° C. (months) | | | | | |
| Assay | | t = 0 | 1 | 3 | 6 | 12 | 18 | 24 |
| Increase in Turbidity (OD 405 nm) | | 0.00 | 0.00 | 0.00 | 0.00 | 0.00 | 0.00 | 0.00 |
| pH | | 5.8 | 5.8 | 5.8 | 5.7 | 5.8 | 5.8 | 5.8 |
| % mAb Recovered by RP-UPLC | | 100 | 100 | 101 | 102 | 115 | 99 | 101 |
| Purity by SE-UPLC | % HMW | 1.1 | 0.8 | 0.7 | 0.9 | 0.7 | 1.0 | 0.5 |
| | % Main | 98.9 | 99.2 | 99.3 | 98.1 | 98.9 | 98.7 | 98.9 |
| | % LMW | 0.00 | 0.00 | 0.00 | 1.0 | 0.4 | 0.2 | 0.7 |
| Charge Variant Analysis | % Acidic | 12.1 | 12.4 | 10.7 | 10.9 | 12.0 | 10.6 | 11.1 |
| by CEX-UPLC | % Main | 38.5 | 38.6 | 35.3 | 39.2 | 38.9 | 32.0 | 35.8 |
| | % Basic | 49.4 | 49.0 | 54.0 | 49.8 | 49.1 | 57.4 | 53.1 |
| MFI (# particles/mL) | 2-10 μm | 546 | 532 | — | 465 | 386 | — | 532 |
| (aspect ratio ≤ 0.85 | ≥10 μm | 29 | 117 | — | 106 | 75 | — | 117 |
| applied) | ≥25 μm | 8 | 8 | — | 8 | 4 | — | 8 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 30 summarizes the collected data of Target Formulation 5 stored with 0.3% phenol at 25° C. for 6 months.

TABLE 30
Target Formulation 5 with 0.3% phenol stored at 25° C. for 6 months

| | Target Formulation | | | | | | |
|---|---|---|---|---|---|---|---|
| | 2 mg/mL mAb C formulation with 0.3% phenol | | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.00 | 0.00 | 0.00 | 0.00 |
| pH | | 5.8 | 5.8 | 5.9 | 5.8 | 5.8 | 5.8 |
| % odronextamab Recovered by RP-UPLC | | 100 | 99 | 100 | 95 | 97 | 102 |
| Purity by SE-UPLC | % HMW | 1.1 | 0.9 | 0.8 | 0.9 | 0.6 | 1.2 |
| | % Main | 98.9 | 99.1 | 99.2 | 99.1 | 99.3 | 98.1 |
| | % LMW | 0.0 | 0.0 | 0.0 | 0.0 | 0.2 | 0.7 |
| Charge Variant Analysis | % Acidic | 12.1 | 13.3 | 13.1 | 15.5 | 12.7 | 17.4 |
| by CEX-UPLC | % Main | 38.5 | 37.4 | 37.9 | 39.2 | 35.9 | 35.6 |
| | % Basic | 49.4 | 49.3 | 49.0 | 45.4 | 51.5 | 47.1 |
| MFI (# particles/mL) | 2-10 μm | 546 | — | 350 | — | — | 302 |
| (aspect ratio ≤ 0.85 | ≥10 μm | 29 | — | 138 | — | — | 33 |
| applied) | ≥25 μm | 8 | — | 31 | — | — | 6 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 31 summarizes the collected data of Target Formulation 5 stored with 0.3% phenol at 40° C. and 45° C. for 3 months.

TABLE 31
Target Formulation 5 with 0.3% phenol stored at 40° C. and 45° C. for 3 months

| | | Target Formulation | | | | | | | | |
|---|---|---|---|---|---|---|---|---|---|---|
| | | 2 mg/mL mAb C formulation with 0.3% phenol | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Pass |
| Increase in Turbidity (OD 405 nm) | | 0.00 | 0.00 | 0.00 | 0.00 | 0.02 | 0.00 | 0.00 | 0.02 | 0.04 |
| pH | | 5.8 | 5.9 | 5.9 | 5.8 | 5.9 | 5.9 | 5.9 | 5.8 | 5.8 |
| % mAb Recovered by RP-UPLC | | 100 | 94 | 100 | 95 | 96 | 100 | 99 | 94 | 93 |
| Purity by SE-UPLC | % HMW | 1.1 | 0.6 | 1.1 | 1.9 | 3.3 | 2.2 | 3.8 | 9.0 | 20.5 |
| | % Main | 98.9 | 99.2 | 98.9 | 98.0 | 95.7 | 97.7 | 96.2 | 90.5 | 78.6 |
| | % LMW | 0.0 | 0.2 | 0.0 | 0.1 | 1.0 | 0.1 | 0.0 | 0.5 | 0.9 |
| Charge Variant Analysis | % Acidic | 12.1 | 23.7 | 29.8 | 42.7 | 36.5 | 29.7 | 40.5 | 58.4 | 30.6 |
| by CEX-UPLC | % Main | 38.5 | 28.2 | 24.5 | 18.5 | 10.0 | 23.5 | 16.7 | 8.0 | 17.7 |
| | % Basic | 49.4 | 48.1 | 45.7 | 38.8 | 53.5 | 46.8 | 42.8 | 33.7 | 51.7 |
| MFI (# particles/mL) | 2-10 μm | 546 | — | 382 | — | 444 | — | 611 | — | NA |
| (aspect ratio ≤ 0.85 | ≥10 μm | 29 | — | 40 | — | 52 | — | 98 | — | NA |
| applied) | ≥25 μm | 8 | — | 10 | — | 0 | — | 17 | — | NA |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Target Formulation 6 is summarized in Tables 32-34.

Table 32 summarizes the collected data of Target Formulation 6 stored with 1% benzyl alcohol at 5° C. for 24 months.

TABLE 32
Target Formulation 6 with 1% benzyl alcohol stored at 5° C. for 24 months

| | | Target Formulation | | | | | | |
|---|---|---|---|---|---|---|---|---|
| | | 2 mg/mL mAb C formulation with 1% benzyl alcohol | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.00 | 0.00 | 0.00 | 0.00 |
| pH | | 5.8 | 5.8 | 5.8 | 5.8 | 5.8 | 5.8 | 5.8 |
| % mAb Recovered by RP-UPLC | | 100 | 102 | 99 | 105 | NA* | 101 | 103 |
| Purity by SE-UPLC | % HMW | 2.0 | 0.8 | 1.4 | 0.9 | 0.5 | 0.9 | 0.3 |
| | % Main | 97.7 | 99.2 | 97.5 | 98.1 | 99.2 | 98.8 | 99.4 |
| | % LMW | 0.3 | 0.0 | 1.1 | 1.0 | 0.3 | 0.4 | 0.3 |
| Charge Variant Analysis | % Acidic | 12.6 | 12.9 | 10.8 | 11.4 | 12.0 | 11.8 | 10.9 |
| by CEX-UPLC | % Main | 37.3 | 37.9 | 35.4 | 38.6 | 38.3 | 34.7 | 35.4 |
| | % Basic | 50.1 | 49.2 | 53.8 | 50.0 | 49.7 | 53.4 | 53.7 |
| MFI (# particles/mL) | 2-10 μm | 240 | 215 | — | 200 | 325 | — | 892 |
| (aspect ratio ≤ 0.85 | ≥10 μm | 40 | 65 | — | 25 | 60 | — | 60 |
| applied) | ≥25 μm | 8 | 6 | — | 8 | 10 | — | 2 |

CEX, cation exchange; HMW, high molecular weight; LMW, low molecular weight; MFI, Micro-Flow Imaging; NA, Not Available (*Data invalid due to error with sample injection); OD, optical density; RH, relative humidity; RP, Reverse Phase; SE, size exclusion; UPLC, ultra-performance liquid chromatography.

Table 33 summarizes the collected data of Target Formulation 6 stored with 1% benzyl alcohol at 25° C. for 6 months.

TABLE 33
Target Formulation 6 with 1% benzyl alcohol stored at 25° C. for 6 months

| | | Target Formulation | | | | | |
|---|---|---|---|---|---|---|---|
| | | 2 mg/mL mAb C formulation with 1% benzyl alcohol | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.00 | 0.00 | 0.00 | 0.00 |
| pH | | 5.8 | 5.8 | 5.8 | 5.8 | 5.8 | 5.8 |
| % mAb Recovered by RP-UPLC | | 100 | 103 | 102 | 98 | 99 | 106 |
| Purity by SE-UPLC | % HMW | 2.0 | 0.7 | 0.7 | 1.4 | 1.8 | 1.0 |
| | % Main | 97.7 | 99.3 | 99.3 | 98.6 | 97.1 | 98.2 |
| | % LMW | 0.3 | 0.0 | 0.0 | 0.0 | 1.2 | 0.9 |
| Charge Variant Analysis | % Acidic | 12.6 | 12.7 | 13.5 | 14.4 | 12.7 | 26.1 |
| by CEX-UPLC | % Main | 37.3 | 38.1 | 38.0 | 41.3 | 34.8 | 26.0 |
| | % Basic | 50.1 | 49.2 | 48.5 | 44.3 | 52.5 | 47.9 |
| MFI (# particles/mL) | 2-10 μm | 200 | — | 407 | — | — | 628 |
| (aspect ratio ≤ 0.85 | ≥10 μm | 25 | — | 65 | — | — | 25 |
| applied) | ≥25 μm | 8 | — | 8 | — | — | 2 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 34 summarizes the collected data of Target Formulation 6 stored with 1% benzyl alcohol at 40° C. and 45° C. for 3 months.

TABLE 34
Target Formulation 6 with 1% benzyl alcohol stored at 40° C. and 45° C. for 3 months

| | | Target Formulation | | | | | | | | |
|---|---|---|---|---|---|---|---|---|---|---|
| | | 2 mg/mL mAb C formulation with 1% benzyl alcohol | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Passs |
| Increase in Turbidity (OD 405 nm) | | 0.00 | 0.00 | 0.00 | 0.00 | 0.02 | 0.00 | 0.01 | 0.03 | 0.07 |
| pH | | 5.8 | 5.8 | 5.9 | 5.8 | 5.8 | 5.8 | 5.9 | 5.8 | 5.7 |
| % mAb Recovered by RP-UPLC | | 100 | 102 | 102 | 97 | 98 | 104 | 103 | 92 | 83 |
| Purity by SE-UPLC | % HMW | 2.0 | 0.7 | 0.7 | 2.8 | 6.9 | 0.7 | 1.3 | 21.3 | 29.3 |
| | % Main | 97.7 | 99.3 | 99.3 | 97.1 | 91.4 | 99.2 | 98.5 | 78.3 | 68.7 |
| | % LMW | 0.3 | 0.0 | 0.0 | 0.2 | 1.7 | 0.0 | 0.2 | 0.4 | 2.0 |
| Charge Variant Analysis | % Acidic | 12.6 | 24.1 | 29.7 | 43.5 | 37.4 | 29.1 | 40.9 | 48.2 | 30.3 |
| by CEX-UPLC | % Main | 37.3 | 27.1 | 23.8 | 17.7 | 10.0 | 24.5 | 15.5 | 7.0 | 18.8 |
| | % Basic | 50.1 | 48.8 | 46.6 | 38.9 | 52.6 | 46.5 | 43.7 | 44.8 | 50.9 |
| MFI (# particles/mL) | 2-10 μm | 240 | — | 215 | — | 325 | — | 281 | — | 1948 |
| (aspect ratio ≤ 0.85 | ≥10 μm | 40 | — | 63 | — | 71 | — | 63 | — | 52 |
| applied) | ≥25 μm | 8 | — | 10 | — | 4 | — | 8 | — | 6 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Target Formulation 7 is summarized in Tables 35-37.

Table 35 summarizes the collected data of Target Formulation 7 stored with 0.3% phenol at 5° C. for 24 months.

TABLE 35
Target Formulation 7 with 0.3% phenol stored at 5° C. for 24 months

| | | Target Formulation | | | | | | |
|---|---|---|---|---|---|---|---|---|
| | | 100 mg/mL mAb C formulation with 0.3% phenol | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.00 | 0.01 | 0.0 | 0.01 |
| pH | | 6.0 | 6.0 | 6.0 | 6.0 | 6.0 | 6.0 | 6.0 |
| % mAb Recovered by RP-UPLC | | 100 | 97 | 94 | 98 | 95 | 92 | 95 |
| Purity by SE-UPLC | % HMW | 1.3 | 1.3 | 1.4 | 1.5 | 1.4 | 1.4 | 1.5 |
| | % Main | 98.7 | 98.5 | 97.4 | 97.0 | 97.7 | 97.8 | 97.8 |
| | % LMW | 0.0 | 0.2 | 0.2 | 1.5 | 0.9 | 0.8 | 0.8 |
| Charge Variant Analysis | % Acidic | 12.5 | 12.8 | 10.4 | 11.0 | 11.9 | 10.5 | 10.6 |
| by CEX-UPLC | % Main | 38.6 | 38.6 | 36.5 | 39.0 | 39.6 | 37.6 | 37.0 |
| | % Basic | 48.9 | 48.6 | 53.0 | 50.0 | 48.5 | 52.0 | 52.4 |
| MFI (# particles/mL) | 2-10 μm | 832 | 861 | — | 678 | 746 | — | 771 |
| (aspect ratio ≤ 0.85 | ≥10 μm | 63 | 148 | — | 58 | 54 | — | 104 |
| applied) | ≥25 μm | 6 | 2 | — | 4 | 4 | — | 6 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 36 summarizes the collected data of Target Formulation 7 stored with 0.3% phenol at 25° C. for 6 months.

TABLE 36
Target Formulation 7 with 0.3% phenol stored at 25° C. for 6 months

| | | Target Formulation | | | | | |
|---|---|---|---|---|---|---|---|
| | | 100 mg/mL mAb C formulation with 0.3% phenol | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.00 | 0.00 | 0.00 | 0.02 |
| pH | | 6.0 | 6.0 | 6.0 | 6.0 | 6.0 | 6.0 |
| % mAb Recovered by RP-UPLC | | 100 | 98 | 99 | 93 | 95 | 98 |
| Purity by SE-UPLC | % HMW | 1.3 | 1.3 | 1.5 | 1.6 | 0.5 | 1.9 |
| | % Main | 98.7 | 98.7 | 98.3 | 97.7 | 99.5 | 96.6 |
| | % LMW | 0.0 | 0.0 | 0.2 | 0.7 | 0.1 | 1.5 |
| Charge Variant Analysis | % Acidic | 12.5 | 12.6 | 13.2 | 14.6 | 13.0 | 26.9 |
| by CEX-UPLC | % Main | 38.6 | 38.6 | 38.5 | 40.9 | 35.2 | 26.7 |
| | % Basic | 48.9 | 48.8 | 48.3 | 44.5 | 51.8 | 46.4 |
| MFI (# particles/mL) | 2-10 μm | 832 | — | 569 | — | — | 765 |
| (aspect ratio ≤ 0.85 | ≥10 μm | 63 | — | 40 | — | — | 38 |
| applied) | ≥25 μm | 6 | — | 4 | — | — | 4 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 37 summarizes the collected data of Target Formulation 7 stored with 0.3% phenol at 40° C. and 45° C. for 3 months.

TABLE 37
Target Formulation 7 with 0.3% phenol stored at 40° C. and 45° C. for 3 months

| | | Target Formulation | | | | | | | | |
|---|---|---|---|---|---|---|---|---|---|---|
| | | 100 mg/mL mAb C formulation with 0.3% phenol | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Passs | Pass | Pass | Pass | Pass | Pass | Pass |
| Increase in Turbidity (OD 405 nm) | | 0.00 | 0.01 | 0.01 | 0.03 | 0.04 | 0.02 | 0.03 | 0.06 | 0.12 |
| pH | | 6.0 | 6.0 | 6.1 | 6.0 | 6.0 | 6.0 | 6.0 | 6.1 | 6.0 |
| % mAb Recovered by RP-UPLC | | 100 | 96 | 100 | 93 | 94 | 102 | 99 | 90 | 88 |
| Purity by SE-UPLC | % HMW | 1.3 | 2.1 | 2.8 | 4.4 | 6.6 | 4.8 | 8.7 | 17.0 | 49.9 |
| | % Main | 98.7 | 97.9 | 97.2 | 94.5 | 92.6 | 94.9 | 91.0 | 81.1 | 48.8 |
| | % LMW | 0.0 | 0.0 | 0.0 | 1.1 | 0.8 | 0.2 | 0.3 | 2.0 | 1.4 |
| Charge Variant Analysis | % Acidic | 12.5 | 23.2 | 29.8 | 43.3 | 34.8 | 29.6 | 41.3 | 47.0 | 32.7 |
| by CEX-UPLC | % Main | 38.6 | 30.3 | 24.5 | 19.3 | 10.2 | 25.0 | 16.1 | 10.8 | 10.0 |
| | % Basic | 48.9 | 46.6 | 45.6 | 37.4 | 54.9 | 45.5 | 42.6 | 42.2 | 57.3 |
| MFI (# particles/mL) | 2-10 μm | 832 | — | 1401 | — | 2491 | — | 707 | — | 1711 |
| (aspect ratio ≤ 0.85 | ≥10 μm | 63 | — | 58 | — | 144 | — | 61 | — | 113 |
| applied) | ≥25 μm | 6 | — | 4 | — | 10 | — | 2 | — | 13 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Target Formulation 8 is summarized in Tables 38-40.

Table 38 summarizes the collected data of Target Formulation 8 stored with 1% benzyl alcohol at 5° C. for 24 months.

TABLE 38
Target Formulation 8 with 1% benzyl alcohol stored at 5° C. for 24 months

| | | Target Formulation | | | | | | |
|---|---|---|---|---|---|---|---|---|
| | | 100 mg/mL mAb C formulation with 1% benzyl alcohol | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.01 | 0.00 | 0.00 | 0.00 |
| pH | | 6.0 | 6.0 | 6.0 | 6.0 | 6.0 | 6.0 | 6.0 |
| % mAb Recovered by RP-UPLC | | 100 | 100 | 99 | 99 | 113 | 98 | 97 |
| Purity by SE-UPLC | % HMW | 1.3 | NA | 1.3 | 1.4 | 1.0 | 1.3 | 1.1 |
| | % Main | 98.5 | NA | 97.8 | 97.8 | 98.0 | 97.5 | 97.7 |
| | % LMW | 0.2 | NA | 0.9 | 0.9 | 1.0 | 1.2 | 1.3 |
| Charge Variant Analysis | % Acidic | 13.0 | 13.0 | 10.5 | 11.1 | 12.0 | 11.0 | 10.4 |
| by CEX-UPLC | % Main | 38.5 | 38.4 | 37.4 | 39.2 | 39.3 | 37.4 | 37.2 |
| | % Basic | 48.5 | 48.6 | 52.1 | 49.8 | 48.8 | 51.5 | 52.4 |
| MFI (# particles/mL) | 2-10 μm | 1364 | 1372 | — | 784 | 2214 | — | 963 |
| (aspect ratio ≤0.85 applied) | ≥10 μm | 38 | 179 | — | 119 | 161 | — | 123 |
| | ≥25 μm | 2 | 6 | — | 15 | 6 | — | 15 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 39 summarizes the collected data of Target Formulation 8 stored with 1% benzyl alcohol at 25° C. for 6 months.

TABLE 39
Target Formulation 8 with 1% benzyl alcohol stored at 25° C. for 6 months

| | | Target Formulation | | | | | |
|---|---|---|---|---|---|---|---|
| | | 100 mg/mL mAb C formulation with 1% benzyl alcohol | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.00 | 0.00 | 0.00 | 0.01 |
| pH | | 6.0 | 6.0 | 6.0 | 6.0 | 6.0 | 6.1 |
| % mAb Recovered by RP-UPLC | | 100 | 100 | 100 | 95 | 96 | 100 |
| Purity by SE-UPLC | % HMW | 1.3 | 1.3 | 1.4 | 1.6 | 2.1 | 2.1 |
| | % Main | 98.5 | 98.5 | 98.5 | 98.3 | 97.3 | 96.8 |
| | % LMW | 0.2 | 0.2 | 0.1 | 0.1 | 0.7 | 1.1 |
| Charge Variant Analysis | % Acidic | 13.0 | 12.9 | 13.3 | 14.6 | 12.5 | 26.1 |
| by CEX-UPLC | % Main | 38.5 | 38.0 | 38.2 | 41.1 | 35.9 | 27.3 |
| | % Basic | 48.5 | 49.1 | 48.5 | 44.3 | 51.6 | 46.6 |
| MFI (# particles/mL) | 2-10 μm | 1364 | — | 1493 | — | — | 621 |
| (aspect ratio ≤0.85 applied) | ≥10 μm | 38 | — | 146 | — | — | 38 |
| | ≥25 μm | 2 | — | 10 | — | — | 10 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 40 summarizes the collected data of Target Formulation 8 stored with 1% benzyl alcohol at 40° C. and 45° C. for 3 months.

TABLE 40
Target Formulation 8 with 1% benzyl alcohol stored at 40° C. and 45° C. for 3 months

| | | Target Formulation | | | | | | | | |
|---|---|---|---|---|---|---|---|---|---|---|
| | | 100 mg/mL mAb C formulation with 1% benzyl alcohol | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Fail |
| Increase in Turbidity (OD 405 nm) | | 0.00 | 0.01 | 0.02 | 0.03 | 0.05 | 0.02 | 0.07 | 0.15 | 0.28 |
| pH | | 6.0 | 6.0 | 6.0 | 6.0 | 6.0 | 6.0 | 6.1 | 6.1 | 6.1 |
| % mAb Recovered by RP-UPLC | | 100 | 101 | 100 | 93 | 94 | 98 | 95 | 87 | 84 |
| Purity by SE-UPLC | % HMW | 1.3 | 1.7 | 1.5 | 8.1 | 13.8 | NA | 4.4 | 38.9 | 36.4 |
| | % Main | 98.5 | 98.0 | 98.1 | 91.6 | 85.0 | NA | 94.7 | 60.4 | 61.0 |
| | % LMW | 0.2 | 0.3 | 0.5 | 0.3 | 1.2 | NA | 0.9 | 0.7 | 2.6 |
| Charge Variant Analysis | % Acidic | 13.0 | 24.0 | 29.3 | 42.8 | 33.0 | 28.2 | 39.3 | 44.9 | 41.1 |
| by CEX-UPLC | % Main | 38.5 | 28.8 | 25.2 | 19.5 | 11.7 | 25.8 | 18.2 | 11.6 | 9.2 |
| | % Basic | 48.5 | 47.2 | 45.6 | 37.7 | 55.3 | 45.9 | 42.5 | 43.5 | 49.8 |
| MFI (# particles/mL) | 2-10 μm | 1364 | — | 1090 | — | 9328 | — | 1093 | — | — |
| (aspect ratio ≤0.85 applied) | ≥10 μm | 38 | — | 202 | — | 337 | — | 233 | — | — |
| | ≥25 μm | 2 | — | 15 | — | 23 | — | 25 | — | — |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

### Control Groups:

Additional testing of four formulations (i.e., Control Formulations 1-4) containing no preservatives were used as a control and showed continued growth for at least one timepoint beyond when full sterilization occurred in the same formulations containing either preservative. Tables 41-52 summarize the collected data points for Control Formulations 1-4 containing no preservatives.

Control Formulation 1 is summarized in Tables 41-43.

Table 41 summarizes the collected data of Control Formulation 1 stored with no preservative at 5° C. for 24 months.

TABLE 41
Control Formulation 1 stored at 5° C. for 24 months

| | | Target Formulation | | | | | | |
|---|---|---|---|---|---|---|---|---|
| | | 2 mg/mL mAb C formulation | | | | | | |
| | | | | Length of Storage at 5° C. (months) | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.00 | 0.00 | 0.00 | 0.00 |
| pH | | 5.8 | 5.9 | 5.8 | 5.8 | 5.8 | 5.8 | 5.8 |
| % mAb Recovered by RP-UPLC | | 100 | 101 | 95 | 105 | NA* | 97 | 104 |
| Purity by SE-UPLC | % HMW | 1.8 | 0.5 | 0.9 | 0.8 | 0.8 | 0.8 | 0.5 |
| | % Main | 98.2 | 99.5 | 99.1 | 99.2 | 99.2 | 99.2 | 99.5 |
| | % LMW | 0.0 | 0.0 | 0.0 | 0.0 | 0.0 | 0.0 | 0.0 |
| Charge Variant Analysis | % Acidic | 11.9 | 13.0 | 10.6 | 10.9 | 11.7 | 10.6 | 10.2 |
| by CEX-UPLC | % Main | 38.8 | 38.2 | 36.5 | 39.7 | 39.6 | 36.6 | 37.5 |
| | % Basic | 49.3 | 48.8 | 53.0 | 49.4 | 48.7 | 52.8 | 52.4 |
| MFI (# particles/mL) | 2-10 μm | 186 | 144 | — | 281 | 225 | — | 632 |
| (aspect ratio ≤0.85 applied) | ≥10 μm | 31 | 29 | — | 21 | 23 | — | 23 |
| | ≥25 μm | 15 | 4 | — | 0 | 4 | — | 4 |

CEX, cation exchange; HMW, high molecular weight; LMW, low molecular weight; MFI, Micro-Flow Imaging; NA, Not Available (*Data invalid due to error with sample injection); OD, optical density; RH, relative humidity; RP, Reverse Phase; SE, size exclusion; UPLC, ultra-performance liquid chromatography.

Table 42 summarizes the collected data of Control Formulation 1 stored with no preservative at 25° C. for 6 months.

TABLE 42
Control Formulation 1 stored at 25° C. for 6 months

| | | Target Formulation | | | | | |
|---|---|---|---|---|---|---|---|
| | | 2 mg/mL mAb C formulation | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.00 | 0.00 | 0.00 | 0.00 |
| pH 2 | | 5.8 | 5.8 | 5.8 | 5.8 | 5.8 | 5.8 |
| % mAb Recovered by RP-UPLC | | 100 | 102 | 101 | 97 | 99 | 106 |
| Purity by SE-UPLC | % HMW | 1.8 | 1.4 | 1.0 | 1.0 | 0.8 | 0.8 |
| | % Main | 98.2 | 98.6 | 99.0 | 99.0 | 99.0 | 97.2 |
| | % LMW | 0.0 | 0.0 | 0.0 | 0.0 | 0.1 | 2.0 |
| Charge Variant Analysis | % Acidic | 11.9 | 12.7 | 12.8 | 13.7 | 11.7 | 23.5 |
| by CEX-UPLC | % Main | 38.8 | 38.1 | 38.4 | 41.6 | 36.0 | 28.7 |
| | % Basic | 49.3 | 49.3 | 48.9 | 44.7 | 52.3 | 47.9 |
| MFI (# particles/mL) | 2-10 μm | 186 | — | 186 | — | — | 423 |
| (aspect ratio ≤0.85 applied) | ≥10 μm | 31 | — | 50 | — | — | 2 |
| | ≥25 μm | 15 | — | 2 | — | — | 0 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 43 summarizes the collected data of Control Formulation 1 stored with no preservative at 40° C. and 45° C. for 3 months.

TABLE 43
Control Formulation 1 stored at 40° C. and 45° C. for 3 months

| | | Target Formulation | | | | | | | | |
|---|---|---|---|---|---|---|---|---|---|---|
| | | 2 mg/mL mAb C formulation | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Pass |
| Increase in Turbidity (OD 405 nm) | | 0.00 | 0.00 | 0.00 | 0.00 | 0.00 | 0.00 | 0.00 | 0.00 | 0.01 |
| pH | | 5.8 | 5.9 | 5.9 | 5.8 | 5.8 | 5.8 | 5.9 | 5.8 | 5.8 |
| % mAb Recovered by RP-UPLC | | 100 | 101 | 101 | 97 | 99 | 102 | 107 | 97 | 96 |
| Purity by SE-UPLC | % HMW | 1.8 | 1.8 | 2.0 | 1.2 | 1.7 | 4.6 | 9.0 | 3.2 | 9.2 |
| | % Main | 98.2 | 98.2 | 97.9 | 98.7 | 97.7 | 95.3 | 90.8 | 963 | 89.7 |
| | % LMW | 0.0 | 0.0 | 0.1 | 0.1 | 0.7 | 0.1 | 0.2 | 0.5 | 1.1 |
| Charge Variant Analysis | % Acidic | 11.9 | 21.5 | 26.5 | 38.5 | 36.2 | 26.3 | 37.4 | 58.4 | 30.6 |
| by CEX-UPLC | % Main | 38.8 | 29.9 | 26.1 | 21.6 | 14.7 | 26.1 | 18.2 | 13.3 | 14.2 |
| | % Basic | 49.3 | 48.7 | 47.4 | 39.9 | 47.1 | 47.6 | 44.4 | 48.2 | 55.2 |
| MFI (# particles/mL) | 2-10 μm | 186 | — | 242 | — | 275 | — | 244 | — | 501 |
| (aspect ratio ≤0.85 applied) | ≥10 μm | 31 | — | 17 | — | 6 | — | 10 | — | 90 |
| | ≥25 μm | 15 | — | 8 | — | 0 | — | 2 | — | 29 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Control Formulation 2 is summarized in Tables 44-46.

Table 44 summarizes the collected data of Control Formulation 2 stored with no preservative at 5° C. for 24 months.

TABLE 44
Control Formulation 2 stored at 5° C. for 24 months

| | | Target Formulation | | | | | | |
|---|---|---|---|---|---|---|---|---|
| | | 100 mg/mL mAb C formulation | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.00 | 0.00 | 0.00 | 0.00 | 0.00 |
| pH | | 5.8 | 5.8 | 5.8 | 5.8 | 5.8 | 5.8 | 5.7 |
| % mAb Recovered by RP-UPLC | | 100 | 99 | 99 | 100 | 96 | 98 | 99 |
| Purity by SE-UPLC | % HMW | 1.3 | 1.1 | 1.4 | 1.3 | 1.3 | 1.4 | 1.5 |
| | % Main | 98.7 | 98.9 | 97.9 | 97.7 | 96.9 | 98.2 | 97.5 |
| | % LMW | 0.0 | 0.0 | 0.7 | 1.0 | 1.8 | 0.4 | 1.0 |
| Charge Variant Analysis | % Acidic | 12.8 | 12.7 | 10.5 | 10.7 | 11.3 | 9.9 | 9.9 |
| by CEX-UPLC | % Main | 38.1 | 38.4 | 36.5 | 39.4 | 39.8 | 38.1 | 38.0 |
| | % Basic | 49.1 | 49.0 | 53.1 | 49.9 | 48.9 | 51.9 | 52.1 |
| MFI (# particles/mL) | 2-10 μm | 1416 | 1040 | — | 1272 | 2100 | — | 94 |
| (aspect ratio ≤0.85 applied) | ≥10 μm | 81 | 150 | — | 144 | 125 | — | 17 |
| | ≥25 μm | 13 | 15 | — | 6 | 8 | — | 6 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 45 summarizes the collected data of Control Formulation 2 stored with no preservative at 25° C. for 6 months.

TABLE 45
Control Formulation 2 stored at 25° C. for 6 months

| | | Target Formulation | | | | | |
|---|---|---|---|---|---|---|---|
| | | 100 mg/mL mAb C formulation | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.01 | 0.00 | 0.00 | 0.01 |
| pH | | 5.8 | 5.8 | 5.8 | 5.8 | 5.8 | 5.8 |
| % mAb Recovered by RP-UPLC | | 100 | 103 | 99 | 95 | 100 | 102 |
| Purity by SE-UPLC | % HMW | 1.3 | 1.3 | 1.3 | 1.5 | 1.7 | 1.9 |
| | % Main | 98.7 | 98.7 | 98.7 | 98.5 | 97.7 | 97.0 |
| | % LMW | 0.0 | 0.0 | 0.0 | 0.0 | 0.6 | 1.1 |
| Charge Variant Analysis | % Acidic | 12.8 | 12.3 | 13.0 | 13.7 | 11.3 | 23.8 |
| by CEX-UPLC | % Main | 38.1 | 38.9 | 38.2 | 41.7 | 35.8 | 29.0 |
| | % Basic | 49.1 | 48.8 | 48.8 | 44.5 | 53.0 | 47.3 |
| MFI (# particles/mL) | 2-10 μm | 1416 | — | 1086 | — | — | 9141 |
| (aspect ratio ≤0.85 applied) | ≥10 μm | 81 | — | 136 | — | — | 175 |
| | ≥25 μm | 13 | — | 17 | — | — | 2 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 46 summarizes the collected data of Control Formulation 2 stored with no preservative at 40° C. and 45° C. for 3 months.

TABLE 46
Control Formulation 2 stored at 40° C. and 45° C. for 3 months

| | | Target Formulation | | | | | | | | |
|---|---|---|---|---|---|---|---|---|---|---|
| | | 100 mg/mL mAb C formulation | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Pass |
| Increase in Turbidity (OD 405 nm) | | 0.00 | 0.01 | 0.02 | 0.02 | 0.04 | 0.01 | 0.03 | 0.05 | 0.11 |
| pH | | 5.8 | 5.8 | 5.8 | 5.8 | 5.8 | 5.8 | 5.8 | 5.8 | 5.9 |
| % mAb Recovered by RP-UPLC | | 100 | 99 | 100 | 95 | 100 | 103 | 98 | 94 | 93 |
| Purity by SE-UPLC | % HMW | 1.3 | 3.0 | 4.7 | 2.9 | 4.5 | 9.4 | 19.8 | 8.8 | 16.4 |
| | % Main | 98.7 | 96.9 | 94.8 | 97.0 | 94.3 | 90.4 | 80.0 | 90.0 | 81.8 |
| | % LMW | 0.0 | 0.0 | 0.5 | 0.1 | 1.2 | 0.2 | 0.2 | 1.3 | 1.8 |
| Charge Variant Analysis | % Acidic | 12.8 | 22.9 | 27.6 | 39.9 | 29.3 | 26.5 | 38.0 | 40.1 | 37.5 |
| by CEX-UPLC | % Main | 38.1 | 28.3 | 25.5 | 20.4 | 11.5 | 26.5 | 17.7 | 12.9 | 8.5 |
| | % Basic | 49.1 | 48.9 | 46.9 | 39.7 | 59.2 | 47.1 | 44.4 | 47.0 | 54.0 |
| MFI (# particles/mL) | 2-10 μm | 1416 | — | 688 | — | 1519 | — | 2578 | — | 738 |
| (aspect ratio ≤0.85 applied) | ≥10 μm | 81 | — | 65 | — | 179 | — | 905 | — | 108 |
| | ≥25 μm | 13 | — | 8 | — | 8 | — | 48 | — | 15 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Control Formulation 3 is summarized in Tables 47-49.

Table 47 summarizes the collected data of Control Formulation 3 stored with no preservative at 5° C. for 24 months.

TABLE 47
Control Formulation 3 stored at 5° C. for 24 months

| | | Target Formulation | | | | | | |
|---|---|---|---|---|---|---|---|---|
| | | 120 mg/mL mAb A + mAb B formulation | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.00 | 0.01 | 0.00 | 0.00 | 0.00 | 0.00 |
| pH | | 6.0 | 6.0 | 6.0 | 6.0 | 6.0 | 6.0 | 6.0 |
| % mAb Recovered by RP-UPLC | | 100 | 98 | 100 | 106 | 102 | 99 | 97 |
| Purity by SE-UPLC | % HMW | 1.0 | 1.3 | 1.0 | 1.3 | 1.1 | 1.3 | 1.2 |
| | % Main | 98.0 | 97.7 | 97.7 | 97.9 | 97.6 | 97.4 | 97.6 |
| | % LMW | 1.0 | 1.0 | 1.3 | 0.8 | 1.3 | 1.3 | 1.2 |
| MFI (# particles/mL) | 2-10 μm | 4253 | 619 | — | 3346 | 4650 | — | 880 |
| (aspect ratio ≤0.85 applied) | ≥10 μm | 252 | 28 | — | 113 | 161 | — | 54 |
| | ≥25 μm | 15 | 8 | — | 16 | 13 | — | 13 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 48 summarizes the collected data of Control Formulation 3 stored with no preservative at 25° C. for 6 months.

TABLE 48
Control Formulation 3 stored at 25° C. for 6 months

| | | Target Formulation | | | | | |
|---|---|---|---|---|---|---|---|
| | | 120 mg/mL mAb A + mAb B formulation | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.00 | 0.00 | 0.01 | 0.01 |
| pH | | 6.0 | 6.0 | 6.1 | 6.0 | 6.0 | 6.0 |
| % mAb Recovered by RP-UPLC | | 100 | 97 | 103 | 99 | 96 | 105 |
| Purity by SE-UPLC | % HMW | 1.0 | 1.2 | 1.4 | 1.4 | 1.5 | 1.7 |
| | % Main | 98.0 | 97.8 | 97.5 | 97.3 | 97.0 | 96.1 |
| | % LMW | 1.0 | 1.1 | 1.1 | 1.3 | 1.3 | 2.1 |
| MFI (# particles/mL) | 2-10 μm | 4253 | — | 2160 | — | — | 1345 |
| (aspect ratio ≤0.85 applied) | ≥10 μm | 252 | — | 220 | — | — | 124 |
| | ≥25 μm | 15 | — | 23 | — | — | 11 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 49 summarizes the collected data of Control Formulation 3 stored with no preservative at 40° C. and 45° C. for 3 months.

TABLE 49
Control Formulation 3 stored at 40° C. and 45° C. for 3 months

| | | Target Formulation | | | | | | | | |
|---|---|---|---|---|---|---|---|---|---|---|
| | | 120 mg/mL mAb A + mAb B formulation | | | | | | | | |
| | | | Length of Storage at 40° C. | | | | Length of Storage at 45° C. | | | |
| | | | (months) | | | | (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Pass |
| Increase in Turbidity (OD 405 nm) | | 0.00 | 0.01 | 0.01 | 0.03 | 0.04 | 0.02 | 0.03 | 0.05 | 0.09 |
| pH | | 6.0 | 6.0 | 6.1 | 6.0 | 6.0 | 6.0 | 6.1 | 6.0 | 6.0 |
| % mAb Recovered by RP-UPLC | | 100 | 103 | 100 | 97 | 95 | 99 | 100 | 96 | 91 |
| Purity by SE-UPLC | % HMW | 1.0 | 1.7 | 2.0 | 2.5 | 4.9 | 2.1 | 2.6 | 3.8 | 7.8 |
| | % Main | 98.0 | 96.9 | 96.2 | 94.1 | 92.1 | 96.2 | 94.9 | 90.8 | 87.3 |
| | % LMW | 1.0 | 1.4 | 1.8 | 3.4 | 3.0 | 1.8 | 2.5 | 5.4 | 4.9 |
| MFI (# particles/mL) | 2-10 μm | 4253 | — | 2232 | — | 4368 | — | 875 | — | 3246 |
| (aspect ratio ≤0.85 applied) | ≥10 μm | 252 | — | 193 | — | 105 | — | 103 | — | 192 |
| | ≥25 μm | 15 | — | 23 | — | 7 | — | 15 | — | 16 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Control Formulation 4 is summarized in Tables 50-52.

Table 50 summarizes the collected data of Control Formulation 4 stored with no preservative at 5° C. for 24 months.

TABLE 50
Control Formulation 4 stored at 5° C. for 24 months

| | | Target Formulation | | | | | | |
|---|---|---|---|---|---|---|---|---|
| | | 200 mg/mL mAb D formulation | | | | | | |
| | | | Length of Storage at 5° C. (months) | | | | | |
| Assay | | t = 0 | 1 | 3 | 6 | 12 | 18 | 24 |
| Visual Appearance | | Pass | Pass | Pass | Pass | Pass | Pass | Pass |
| Increase in Turbidity (OD 405 nm) | | 0.00 | 0.01 | 0.00 | 0.00 | 0.03 | 0.00 | 0.01 |
| pH | | 5.8 | 5.9 | 5.8 | 5.8 | 5.9 | 5.8 | 5.8 |
| % mAb D Recovered by RP-UPLC | | 100 | 101 | 92 | 111 | 97 | 103 | 107 |
| Purity by SE-UPLC | % HMW | 0.9 | 0.9 | 0.8 | 1.0 | 0.8 | 0.8 | 0.8 |
| | % Main | 98.8 | 98.8 | 98.9 | 98.0 | 98.8 | 98.8 | 98.5 |
| | % LMW | 0.3 | 0.3 | 0.3 | 1.0 | 0.4 | 0.4 | 0.7 |
| Charge Variant Analysis | % Acidic | 21.8 | 21.8 | 22.1 | 17.9 | 21.8 | 22.4 | 22.3 |
| by CEX-UPLC | % Main | 61.7 | 61.6 | 62.3 | 61.9 | 63.0 | 63.3 | 63.6 |
| | % Basic | 16.6 | 16.7 | 15.6 | 16.1 | 15.2 | 14.4 | 14.1 |
| MFI (# particles/mL) | 2-10 μm | 4616 | 7627 | — | 646 | 3167 | — | 3330 |
| (aspect ratio ≤0.85 applied) | ≥10 μm | 286 | 578 | — | 63 | 169 | — | 292 |
| | ≥25 μm | 17 | 52 | — | 8 | 15 | — | 15 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 51 summarizes the collected data of Control Formulation 4 stored with no preservative at 25° C. for 6 months.

TABLE 51
Control Formulation 4 stored at 25° C. for 6 months

| | | Target Formulation | | | | | |
|---|---|---|---|---|---|---|---|
| | | 200 mg/mL mAb D formulation | | | | | |
| | | | Length of Storage at 25° C. (months) | | | | |
| Assay | | t = 0 | 0.5 | 1 | 2 | 3 | 6 |
| Visual Appearance | | Pass | Pass | Pass | Pass | Pass | Pass |
| Increase in Turbidity (OD 405 nm) | | 0.00 | 0.00 | 0.00 | 0.00 | 0.00 | 0.02 |
| pH | | 5.8 | 5.8 | 5.9 | 5.9 | 5.8 | 5.8 |
| % mAb Recovered by RP-UPLC | | 100 | 103 | 102 | 97 | 93 | 112 |
| Purity by SE-UPLC | % HMW | 0.9 | 1.0 | 1.0 | 1.1 | 1.0 | 1.1 |
| | % Main | 98.8 | 98.7 | 98.7 | 98.5 | 98.7 | 97.6 |
| | % LMW | 0.3 | 0.3 | 0.3 | 0.4 | 0.4 | 1.3 |
| Charge Variant Analysis | % Acidic | 21.8 | 21.6 | 21.5 | 22.2 | 22.6 | 21.5 |
| by CEX-UPLC | % Main | 61.7 | 61.7 | 62.1 | 63.2 | 63.7 | 61.1 |
| | % Basic | 16.6 | 16.8 | 16.3 | 14.6 | 13.7 | 14.7 |
| MFI (# particles/mL) | 2-10 μm | 4616 | — | 4368 | — | — | 440 |
| (aspect ratio ≤0.85 applied) | ≥10 μm | 286 | — | 386 | — | — | 69 |
| | ≥25 μm | 17 | — | 98 | — | — | 6 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

Table 52 summarizes the collected data of Control Formulation 4 stored with no preservative at 40° C. and 45° C. for 3 months.

TABLE 52
Control Formulation 4 stored at 40° C. and 45° C. for 3 months

| | | Target Formulation | | | | | | | | |
|---|---|---|---|---|---|---|---|---|---|---|
| | | 200 mg/mL mAb D formulation | | | | | | | | |
| | | | Length of Storage at | | | | Length of Storage at | | | |
| | | | 40° C. (months) | | | | 45° C. (months) | | | |
| Assay | | t = 0 | 0.5 | 1 | 2 | 3 | 0.5 | 1 | 2 | 3 |
| Visual Appearance | | Pass | Pass | Pass | Pass | Pass | Pass | Pass | Pass | Pass |
| Increase in Turbidity (OD 405 nm) | | 0.00 | 0.01 | 0.01 | 0.02 | 0.03 | 0.02 | 0.02 | 0.05 | 0.08 |
| pH | | 5.8 | 5.8 | 5.9 | 5.8 | 5.8 | 5.9 | 5.9 | 5.9 | 5.9 |
| % mAb Recovered by RP-UPLC | | 100 | 104 | 101 | 96 | 93 | 108 | 101 | 94 | 90 |
| Purity by SE-UPLC | % HMW | 0.9 | 1.4 | 1.8 | 2.8 | 4.4 | 3.0 | 5.4 | 11.1 | 17.1 |
| | % Main | 98.8 | 98.2 | 97.6 | 96.5 | 94.7 | 96.4 | 93.7 | 87.9 | 73.7 |
| | % LMW | 0.3 | 0.4 | 0.6 | 0.7 | 1.0 | 0.6 | 0.9 | 1.0 | 9.3 |
| Charge Variant Analysis | % Acidic | 21.8 | 22.6 | 25.0 | 34.2 | 42.8 | 23.6 | 31.8 | 46.3 | 59.4 |
| by CEX-UPLC | % Main | 61.7 | 61.2 | 59.4 | 50.3 | 42.6 | 61.2 | 53.0 | 39.5 | 9.7 |
| | % Basic | 16.6 | 16.2 | 15.6 | 15.5 | 14.6 | 15.3 | 15.3 | 14.2 | 30.9 |
| MFI (# particles/mL) | 2-10 μm | 4616 | — | 6218 | — | 1785 | — | 14817 | — | 1522 |
| (aspect ratio ≤0.85 applied) | ≥10 μm | 286 | — | 466 | — | 136 | — | 1437 | — | 108 |
| | ≥25 μm | 17 | — | 100 | — | 29 | — | 290 | — | 25 |

CEX, cation exchange; HMW, high molecular weight; LMW, low molecular weight; MFI, Micro-Flow Imaging; NA, Not Available; OD, optical density; RH, relative humidity; RP, Reverse Phase; SE, size exclusion; UPLC, ultra-performance liquid chromatography.

### CONCLUSION OF ADDITIONAL TESTING

The sample formulations with preservatives were compared to the control formulations without preservatives to evaluate the effect of preservatives on protein stability.

At the stress conditions (40° C. and 45′C), preservatives increased mAb aggregation as detected by SE-UPLC in some of the sample formulations compared to the control formulations. Preservatives did not influence protein charge or subvisible particle formation even in stress conditions. However, stability of formulations of mAbs in the presence of preservatives was comparable to controls at 5° C. for at least 24 months and at and 25° C. for at least 6 months. Overall, the formulations with preservatives were stable under the proposed storage conditions at 5° C. for at least 24 months and at 25° C. for at least 6 months.

While in the foregoing specification this invention has been described in relation to certain aspects thereof, and many details have been put forth for the purpose of illustration, it will be apparent to those skilled in the art that the invention is susceptible to additional aspects and that certain of the details described herein can be varied considerably without departing from the basic principles of the invention.

1. A multiple-dose container comprising a parenteral Fc-containing protein preparation, wherein the preparation comprises in an aqueous solution
(a) at least one type of Fc-containing protein, and
(b) phenol or benzyl alcohol.

2. The multiple-dose container of claim 1, wherein the Fc-containing proteins are at a concentration of 0.1 mg/ml to 500 mg/ml.

3. The multiple-dose container of claim 1, wherein the preparation comprises phenol at a concentration of 1 mg/ml to 10 mg/ml, 2 mg/ml to 5 mg/ml, or 3 mg/ml.

4. (canceled)

5. (canceled)

6. The multiple-dose container of claim 1, wherein the preparation comprises benzyl alcohol at a concentration of 1 mg/ml to 15 mg/ml, 3 mg/ml to 12 mg/ml, or 10 mg/ml.

7. (canceled)

8. (canceled)

9. The multiple-dose container of claim 1, wherein the container is a single patient use container.

10. The multiple-dose container of claim 1, wherein the container has a capacity of 1 ml to 100 ml, 5 ml to 100 ml, 10 ml to 50 ml, 20 ml to 40 ml, or 30 ml.

11. (canceled)

12. (canceled)

13. (canceled)

14. (canceled)

15. The multiple-dose container of claim 1, wherein the Fc-containing protein is a monoclonal antibody.

16. The multiple-dose container of claim 15, wherein the monoclonal antibody is a bispecific antibody.

17. The multiple-dose container of claim 1, wherein the container comprises two or more types of Fc-containing proteins.

18. The multiple-dose container of claim 1, wherein the Fc-containing protein is a receptor Fc-fusion protein.

19. The multiple-dose container of claim 1, wherein the Fc-containing protein is a trap protein.

20. The multiple-dose container of claim 1, wherein the preparation comprises phenol.

21. The multiple-dose container of claim 1, wherein the preparation comprises benzoyl alcohol.

22. A parenteral Fc-containing protein preparation comprising in an aqueous solution
(a) at least one type of Fc-containing protein, and
(b) phenol or benzyl alcohol.

23. The parenteral Fc-containing protein preparation of claim 22, wherein the Fc-containing proteins are at a concentration of 0.1 mg/ml to 500 mg/ml.

24. The parenteral Fc-containing protein preparation of claim 22, wherein the preparation comprises phenol at a concentration of 1 mg/ml to 10 mg/ml, 2 mg/ml to 5 mg/ml, or 3 mg/ml.

25. (canceled)

26. (canceled)

27. The parenteral Fc-containing protein preparation of claim 22, wherein the preparation comprises benzyl alcohol at a concentration of 1 mg/ml to 15 mg/ml, 2 mg/ml to 12 mg/ml, or 10 mg/ml.

28. (canceled)

29. (canceled)

30. The parenteral Fc-containing protein preparation of claim 22, wherein the Fc-containing protein is a monoclonal antibody.

31. The parenteral Fc-containing protein preparation of claim 30, wherein the monoclonal antibody is a bispecific antibody.

32. The parenteral Fc-containing protein preparation of claim 22, wherein the preparation comprises two types of Fc-containing proteins.

33. The parenteral Fc-containing protein preparation of claim 22, wherein the Fc-containing protein is a receptor Fc-fusion protein.

34. The parenteral Fc-containing protein preparation of claim 22, wherein the Fc-containing protein is a trap protein.

35. The parenteral Fc-containing protein preparation of claim 22, wherein the preparation comprises phenol.

36. The parenteral Fc-containing protein preparation of claim 22, wherein the preparation comprises benzyl alcohol.

37. A method of stabilizing an Fc-containing protein preparation comprising the steps of:
providing an Fc-containing protein in an aqueous solution comprising phenol or benzyl alcohol; and
filling a container with the Fc-containing protein in an aqueous solution comprising phenol or benzyl alcohol.

38. The method according to claim 37, wherein the phenol or benzyl alcohol is added to an aqueous solution comprising the Fc-containing protein.

39. The method according to claim 37, wherein the Fc-containing protein is at a concentration of 0.1 mg/ml to 500 mg/ml.

40. The method according to claim 37, wherein the aqueous solution comprises phenol at a concentration of 1 mg/ml to 10 mg/ml, 2 mg/ml to 5 mg/ml, or 3 mg/ml.

41. (canceled)

42. (canceled)

43. The method according to claim 37, wherein the aqueous solution comprises benzyl alcohol at a concentration of 1 mg/ml to 15 mg/ml, 3 mg/ml to 12 mg/ml, or 10 mg/ml.

44. (canceled)

45. (canceled)

46. The method according to claim 37, wherein the Fc-containing protein is a monoclonal antibody.

47. The method according to claim 46, wherein the monoclonal antibody is a bispecific antibody.

48. The method according to claim 37, wherein the aqueous solution comprises two or more types of Fc-containing proteins.

49. The method according to claim 37, wherein the Fc-containing protein is a receptor Fc-fusion protein.

50. The method according to claim 37, wherein the Fc-containing protein is a trap protein.

51. The method according to claim 37, wherein the aqueous solution comprises phenol.

52. The method according to claim 37, wherein the aqueous solution comprises benzyl alcohol.
